SEQUENCE LISTING (45 sequences)

SEQ ID NO: 1 - DNA, 26 bp, synthetic construct
  source 1..26: mol_type = other DNA
acaggtacgttaatagttaatagcgt

SEQ ID NO: 2 - DNA, 22 bp, synthetic construct
  source 1..22: mol_type = other DNA
atattgcagcagtacgcacaca

SEQ ID NO: 3 - DNA, 26 bp, synthetic construct
  source 1..26: mol_type = other DNA
acactagccatccttactgcgcttcg

SEQ ID NO: 4 - protein, 6 aa, SARS-CoV-2
PRRARS

SEQ ID NO: 5 - protein, 6 aa, synthetic construct
PGSASS

SEQ ID NO: 6 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - DNA, 3822 bp, synthetic construct
  source 1..3822: mol_type = other DNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 8 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - DNA, 3822 bp, synthetic construct
  source 1..3822: mol_type = other DNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggctctgcctctagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccacccgaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 10 - protein, 1271 aa, SARS-CoV-2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 3816 bp, synthetic construct
  source 1..3816: mol_type = other DNA
atgttcgtcttcctggtcctgctgcctctggtctcctcacagtgcgtcaatctgagaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgaggtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcggtaccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcgcagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 12 - protein, 1271 aa, SARS-CoV-2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 3816 bp, synthetic construct
  source 1..3816: mol_type = other DNA
atgttcgtcttcctggtcctgctgcctctggtctcctcacagtgcgtcaatctgagaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgaggtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcggtaccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcgcggctctgcctctagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaatgtggtgaatcagaacgcccaggccctgaataccctggtgaagcagctgagcagcaacttcggcgccatctctagcgtgctgaatgacatcctgagccggctggacccgccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 14 - protein, 1270 aa, SARS-CoV-2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3813 bp, synthetic construct
  source 1..3813: mol_type = other DNA
atgttcgtgttcctggtgctgctgcccctggtgagcagccagtgcgtgaaccttacaaccaggacccagctgcctcccgcctacaccaacagcttcacaaggggcgtttactaccccgacaaggtgttcaggagcagcgtgctgcacagcacccaggacctgttcctgcccttcttcagcaacgtgacctggttccacgtgatcagcggcaccaacggcaccaagaggttcgacaaccccgtgctgcccttcaacgacggcgtgtacttcgccagcatcgagaagagcaacatcatcaggggctggatcttcggcaccaccctggacagcaagacccagagccttctgatcgtgaacaacgccaccaacgtggtgatcaaggtgtgcgagttccagttctgcaacgaccccttcctggaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcatcgtgagggagcccgaggacctgcctcagggattctctgccctggaacctttggtggacctgcccatcggcatcaacatcaccaggttccagaccctgctggccctgcacaggagctacctgacacctggagacagcagctctggctggacagctggagccgctgcttactacgtgggctacctgcaacctaggaccttcctgctgaagtacaacgagaacggcaccatcaccgacgccgtggactgcgcccttgatcctctgtctgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcttggaacaggaagaggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatcaggggcgatgaagtgaggcagatcgcccctggccagaccggaaacatcgctgactacaattacaagctgcccgacgacttcaccggctgcgtgatcgcctggaacagcaacaagctggacagcaaggtgagcggcaactataactacctgtacaggctgttcaggaagagcaacctgaagcccttcgagagggacatcagcaccgagatctaccaggccggcaacaagccctgtaatggagtggccggcttcaactgctacttccccttgaggagctacagcttcaggcccacctacggcgttggacaccagccttacagggtggtggtgctgtctttcgagctgctgcatgcccctgccaccgtttgcggacccaagaagagcaccaacctggtgaagaacaagtgcgtgaatttcaacttcaatggcctgaagggcaccggcgtgctgaccgagagcaacaagaagttcctgcccttccagcagttcggcagggacatcgccgacaccacagatgctgtgagggaccctcagacactggagatcctggacatcaccccttgcagcttcggcggcgtgtccgtgattacccctggcaccaacacaagcaaccaggtggccgtgctgtaccagggcgttaactgcaccgaggtgcctgttgccatccacgccgaccagttaacccctacctggagggtgtacagcaccggcagcaacgttttccagacaagggccggctgcctgattggcgctgagtacgtgaacaacagctacgagtgcgacatccccatcggcgccggcatctgcgcctcttatcaaacccagaccaagagccacaggagggccaggagcgtggccagccaaagcattatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggaagcttctgcacccagctgaagagggccctgaccggcattgccgttgagcaggacaagaacacccaggaggtgttcgcccaggtgaagcagatctacaagacccctcccatcaagtacttcggcggcttcaacttcagccagatcctgcccgaccccagcaagccctctaagaggagcttcatcgaggacctgctgttcaacaaggtgaccctggccgacgccggcttcatcaagcagtacggagactgcttgggagacatcgccgccagggacctgatctgcgctcagaagttcaagggcctgaccgtgttgcctcccctgctgaccgacgagatgatcgctcagtacaccagcgccctgttggccggcaccattacatctggctggacatttggagccggcgccgccttacagatcccctttgccatgcagatggcttacaggttcaacggcatcggcgtgacccagaacgtgctgtacgagaaccagaagctgatcgccaaccagttcaacagcgccatcggcaagatccaggatagcctgagcagcaccgcttctgccctgggcaagctgcaggatgtggtgaaccacaacgcccaggcccttaacaccctggtgaagcagctgagcagcaagttcggcgccatcagcagcgtgctgaacgacatcttcagcaggctggacaaggtggaggccgaggtgcagatcgacaggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 16 - protein, 1270 aa, SARS-CoV-2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - DNA, 3813 bp, synthetic construct
  source 1..3813: mol_type = other DNA
atgttcgtgttcctggtgctgctgcccctggtgagcagccagtgcgtgaaccttacaaccaggacccagctgcctcccgcctacaccaacagcttcacaaggggcgtttactaccccgacaaggtgttcaggagcagcgtgctgcacagcacccaggacctgttcctgcccttcttcagcaacgtgacctggttccacgtgatcagcggcaccaacggcaccaagaggttcgacaaccccgtgctgcccttcaacgacggcgtgtacttcgccagcatcgagaagagcaacatcatcaggggctggatcttcggcaccaccctggacagcaagacccagagccttctgatcgtgaacaacgccaccaacgtggtgatcaaggtgtgcgagttccagttctgcaacgaccccttcctggaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcatcgtgagggagcccgaggacctgcctcagggattctctgccctggaacctttggtggacctgcccatcggcatcaacatcaccaggttccagaccctgctggccctgcacaggagctacctgacacctggagacagcagctctggctggacagctggagccgctgcttactacgtgggctacctgcaacctaggaccttcctgctgaagtacaacgagaacggcaccatcaccgacgccgtggactgcgcccttgatcctctgtctgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcttggaacaggaagaggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatcaggggcgatgaagtgaggcagatcgcccctggccagaccggaaacatcgctgactacaattacaagctgcccgacgacttcaccggctgcgtgatcgcctggaacagcaacaagctggacagcaaggtgagcggcaactataactacctgtacaggctgttcaggaagagcaacctgaagcccttcgagagggacatcagcaccgagatctaccaggccggcaacaagccctgtaatggagtggccggcttcaactgctacttccccttgaggagctacagcttcaggcccacctacggcgttggacaccagccttacagggtggtggtgctgtctttcgagctgctgcatgcccctgccaccgtttgcggacccaagaagagcaccaacctggtgaagaacaagtgcgtgaatttcaacttcaatggcctgaagggcaccggcgtgctgaccgagagcaacaagaagttcctgcccttccagcagttcggcagggacatcgccgacaccacagatgctgtgagggaccctcagacactggagatcctggacatcaccccttgcagcttcggcggcgtgtccgtgattacccctggcaccaacacaagcaaccaggtggccgtgctgtaccagggcgttaactgcaccgaggtgcctgttgccatccacgccgaccagttaacccctacctggagggtgtacagcaccggcagcaacgttttccagacaagggccggctgcctgattggcgctgagtacgtgaacaacagctacgagtgcgacatccccatcggcgccggcatctgcgcctcttatcaaacccagaccaagagccacggatccgcctctagcgtggccagccaaagcattatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggaagcttctgcacccagctgaagagggccctgaccggcattgccgttgagcaggacaagaacacccaggaggtgttcgcccaggtgaagcagatctacaagacccctcccatcaagtacttcggcggcttcaacttcagccagatcctgcccgaccccagcaagccctctaagaggagcttcatcgaggacctgctgttcaacaaggtgaccctggccgacgccggcttcatcaagcagtacggagactgcttgggagacatcgccgccagggacctgatctgcgctcagaagttcaagggcctgaccgtgttgcctcccctgctgaccgacgagatgatcgctcagtacaccagcgccctgttggccggcaccattacatctggctggacatttggagccggcgccgccttacagatcccctttgccatgcagatggcttacaggttcaacggcatcggcgtgacccagaacgtgctgtacgagaaccagaagctgatcgccaaccagttcaacagcgccatcggcaagatccaggatagcctgagcagcaccgcttctgccctgggcaagctgcaggatgtggtgaaccacaacgcccaggcccttaacaccctggtgaagcagctgagcagcaagttcggcgccatcagcagcgtgctgaacgacatcttcagcaggctggaccctccggaggccgaggtgcagatcgacaggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 18 - protein, 567 aa, Influenza A virus
MEKIVLLLAVISLVKSDQICIGYHANNSTKQVDTIMEKNVTVTHAQDILEKTHNGKLCDLNGVKPLILKDCSVAGWLLGNPMCDEFIRVPEWSYIVERANPANDLCYPGTLNDYEELKHLLSRINHFEKTLIIPRSSWPNHETSLGVSAACPYQGASSFFRNVVWLIKKDDAYPTIKISYNNTNREDLLILWGIHHSNNAAEQTNLYKNPDTYVSVGTSTLNQRLVPKIATRSQVNGQSGRMDFFWTILKPNDAIHFESNGNFIAPEYAYKIVKKGDSTIMKSEMEYGHCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPKYSEEAILKREEISGVKLESIGTYQILSIYSTVASSLALAIIVAGLSLWMCSNGSLQCRICI

SEQ ID NO: 19 - DNA, 1704 bp, synthetic construct
  source 1..1704: mol_type = other DNA
atggaaaaaatcgttctcctgctggcggttatctccctcgtcaaatcggaccaaatctgtatcggttaccacgcaaacaactctacgaaacaggtggacactatcatggaaaagaacgttaccgtgactcacgctcaagacatcctggagaaaacacataacggaaagctctgcgacttgaacggtgtgaaacctttgatcctgaaggattgctccgtggctggttggctgctgggaaacccaatgtgtgacgagttcatcagagtcccggaatggagctacattgttgagcgcgcaaaccctgcgaacgacctctgctaccccggcaccttgaacgattacgaggaactgaagcacttgctgtctcgtatcaaccatttcgaaaaaacactcatcattcctaggtccagctggcccaaccacgagacgagtttgggagtctcggctgcctgtccataccagggtgcatcttcattcttccgtaacgtggtctggctgatcaagaaagacgatgcgtacccgacaatcaagattagttacaacaacacgaacagggaagacctcttgatcctgtggggcattcaccattccaacaacgcagcggagcagaccaacctctacaagaacccagatacttacgtctccgttggaacaagcacgctgaaccaacgtctcgttccgaagatcgcaacaaggtcccaggtgaacggtcaatccggtcgcatggacttcttctggacgatcctgaagcctaacgatgcgattcacttcgaatcaaacggtaacttcatcgctcccgagtacgcctacaagattgtgaagaaaggcgactctactatcatgaagtcagagatggaatacggccactgcaacaccaagtgtcagactcccatcggagctattaacagttcgatgccattccacaacatccatccgctcacaattggagagtgccctaagtacgttaaaagtaacaagctcgtgttggccaccggcctgagaaactcgcccctgagggaacgccgtaggaagaggggattgttcggtgcaatcgcgggtttcattgagggtggctggcagggaatggtggacggatggtacggataccaccatagtaacgaacagggatcgggttacgctgccgacaaagagtctacacaaaaggctatcgatggcgtgacgaacaaagtcaactcaatcattgacaagatgaacacccaattcgaggccgtcggaagagagttcaacaacctggagagacgcatcgaaaacttgaacaagaaaatggaagacggtttcctcgatgtctggacctacaacgctgagctgctcgttctgatggagaacgaacgcactctcgacttccacgattccaacgtgaaaaacttgtacgacaaggtccgtctgcaactcagggataacgccaaggaactgggcaacggatgtttcgagttctaccataagtgcgacaacgagtgtatggaatccgtgagaaacggtacttacgattacccaaaatacagcgaggaagcaatcctgaagcgcgaggaaatttctggcgttaaattggagtcaatcggaacctaccaaatcctgtctatttactcaactgtcgcctccagcttggctctggccatcattgtggcgggtctgtccctctggatgtgttcaaacggtagcctgcaatgtcgtatctgtatctaa

SEQ ID NO: 20 - protein, 469 aa, Influenza A virus
MNPNQKIITIGSVSLTIATVCFLMQIAILATTVTLHFKQNECSIPSNNQVVPCEPIIIERNITEIVYLNNTIIEKELCPKLAEYRDWSKPQCQITGFAPFSKDNSIRLSAGGDIWVTREPYVSCSPNKCYQFALGQGTTLDNKHSNGTIHDRIPHRTLLMNELGVPFHLGTKQVCIAWSSSSCHDGRAWLHICVTGDDRNATASFIYNGVLVDSISSWSQNILRTQESECVCINGTCTVVMTDGSASGRADTRVLFIKEGKIVHISPLSGSAQHIEECSCYPRYPDVRCVCRDNWKGSNRPVIDISMADYSIDSSYVCSGLVGDTPRNDDSSSNSNCKDPNNERGSPGVKGWAFDYGNDVWMGRTISKDSRSGYETFRVIDGWTTANSKSQVNRQVIVDNNNWSGYSGIFSVEGKSCINRCFYVELIRGRPQETRVWWTSNSIVVFCGTSGTYGTGSWPDGANINFMPI

SEQ ID NO: 21 - DNA, 1410 bp, synthetic construct
  source 1..1410: mol_type = other DNA
atgaatcctaatcaaaaaatcatcacaataggtagtgtttcgctgaccatagcaaccgtctgctttttgatgcaaatcgcaatcttggcaaccacggttaccttgcacttcaagcagaatgagtgttcgattccgagtaacaatcaagtggtgccctgcgagccgattatcattgaaaggaacataactgaaatagtgtacttgaacaatactataattgagaaggaactgtgccctaaactcgccgaataccgggactggagcaaaccccagtgtcagattactggctttgcgccattctcgaaagacaacagcattcggctctccgcaggaggtgacatctgggtgacgcgagagccttatgtctcctgttccccgaacaagtgttatcagttcgcgctcggccaaggaacgaccctggataataaacacagtaacggaaccattcatgaccgtattccacaccgcacgctcctcatgaatgagctgggtgtgcccttccatctcggcacaaaacaggtatgtatcgcgtggagcagttcctcctgtcatgacggacgggcatggctgcacatttgtgtcacgggtgatgaccgcaatgccacggccagcttcatatacaatggcgtgttggtggacagtatttcgtcgtggagtcaaaacatcctcaggacacaggagagtgaatgtgtttgtatcaacggaacctgcacagttgtaatgaccgacggatcggctagtggtagggcagatactcgcgttttgtttatcaaggaaggcaaaatcgtgcacattagtcctttgagcggctccgcccaacatatcgaggagtgctcctgctacccccgctatccagacgttcggtgtgtatgtagggataattggaagggctcgaaccgcccggttatagatattagcatggccgattactcgatagacagcagttatgtatgctcgggactggtgggagatacgccgaggaacgacgacagctcgtccaatagtaattgcaaagatccgaataacgagcggggcagtcccggtgttaaaggatgggcatttgactacggcaatgatgtgtggatgggacgaaccataagtaaggatagccgtagcggctacgagacttttcgggtaattgatggctggaccacggctaattccaagtcgcaagttaatcgccaggtaatagttgataacaataattggtcgggatactcgggtattttttccgtcgaaggaaaaagctgcattaacaggtgcttctacgttgaattgattaggggtcgtcctcaggagacacgtgtctggtggactagcaatagtatagtcgtgttctgtggcacgtccggaacgtatggaaccggatcgtggcctgacggagcaaatattaatttcatgcccatctaa

SEQ ID NO: 22 - protein, 254 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIRHENRMVLASTTAKAMEQMAGSSEQAAEAMEIASQARQMVQAMRAIGTHPSSSTGLRDDLLENLQTYQKRMGVQMQRFKGS

SEQ ID NO: 23 - DNA, 765 bp, synthetic construct
  source 1..765: mol_type = other DNA
atgagcctgctgactgaagtggaaacctatgtcctgagtattgtcccaagcggccccctgaaagccgagattgcacagagactggaagacgtgttcgcaggaaagaacaccgatctggaggccctgatggaatggctgaagacacgccccatcctgtcacctctgactaaaggcatcctgggcttcgtgtttaccctgacagtcccaagcgagcggggcctccagcggagaaggttcgtgcagaatgctctgaacggcaatggggaccccaacaatatggataaggccgtcaaactgtaccggaagctgaaaagagagatcacctttcacggcgcaaaggaaattgccctgagctacagcgccggcgccctggccagctgcatgggactgatctataacagaatgggcgctgtgaccacagaggtcgcatttgggctggtgtgcgccacatgtgaacagatcgctgactctcagcaccgcagtcatcgacagatggtggccactaccaaccctctgattaggcacgagaatcgcatggtcctggcctccacaactgcaaaagctatggagcagatggctgggagctccgaacaggccgctgaggcaatggaaatcgcttctcaggcacgacagatggtgcaggccatgagggctattgggactcatccctctagttcaaccggactgagggacgatctgctggagaacctgcagacctatcagaagaggatgggagtccagatgcagagattcaagggatcctga

SEQ ID NO: 24 - protein, 97 aa, Influenza A virus
MSLLTEVETPIRNEWGCRCNDSSDPLVVAANIIGILHLILWILDRLFFKCVYRLFKHGLKRGPSTEGVPESMREEYRKEQQNAVDADDSHFVSIELE

SEQ ID NO: 25 - DNA, 294 bp, synthetic construct
  source 1..294: mol_type = other DNA
atgtccctgctgaccgaagtggaaactcctattagaaacgaatggggctgccgatgtaacgactcctctgaccctctggtggtggctgccaacatcattggaatcctgcacctgatcctgtggattctggacaggctgttctttaagtgcgtgtaccgcctgttcaagcatggcctgaaaagaggccccagcaccgagggcgtccctgaatccatgcgggaggaatatagaaaagagcagcagaacgccgtggacgccgatgatagccattttgtgagcattgaactggaataa

SEQ ID NO: 26 - protein, 218 aa, Escherichia coli
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSGSREFRSY

SEQ ID NO: 27 - protein, 132 aa, Borrelia coriaceae
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 28 - protein, 239 aa, synthetic construct
MVSKGEELFTGVVPILVELDGDVNGHKFSVSGEGEGDATYGKLTLKFICTTGKLPVPWPALVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYK

SEQ ID NO: 29 - protein, 19 aa, synthetic construct
ATNFSLLKQAGDLEENPGP

SEQ ID NO: 30 - DNA, 654 bp, synthetic construct
  source 1..654: mol_type = other DNA
atgtcaaggctggataaatcaaaggtcatcaactccgctctggaactgctgaacgaagtgggaatcgaggggctgactacaaggaaactggcccagaagctgggcgtcgagcagcctaccctgtactggcacgtgaagaacaaacgcgctctgctggacgccctggctatcgaaatgctggatcgacaccatacacatttctgcccactggagggggaaagctggcaggacttcctgcggaacaatgctaagagcttccggtgtgcactgctgtcccacagggatggagctaaggtccatctgggcactcggcccaccgagaaacagtacgagaccctggaaaaccagctggccttcctgtgccagcagggcttttctctggaaaatgcactgtatgccctgagtgctgtgggccacttcacactggggtgcgtgctggaggaccaggaacatcaggtggcaaaggaggaaagagagactccaaccacagactctatgccccctctgctgcggcaggccattgagctgtttgatcaccagggcgcagaacccgccttcctgtttgggctggagctgatcatttgcggactggaaaaacagctgaaatgtgaaagcgggagcgcctacagtgggtccagagagttccgatcctac

SEQ ID NO: 31 - DNA, 399 bp, synthetic construct
  source 1..399: mol_type = other DNA
atggcaaaaccactgtcacaggaagaatccacactgatcgagcgggcaactgcaactattaactctatccctattagcgaagactactctgtggctagtgccgctctgagctccgacggaaggatcttcaccggcgtgaacgtctaccacttcaccggcggcccctgcgcagagctggtggtcctgggcaccgccgccgccgccgccgccggcaatctgacctgtatcgtggccattgggaacgaaaatcgaggcatcctgagcccatgcgggcggtgtagacaggtgctgctggatctgcatcctggaatcaaggccattgtcaaagactctgatggccagcctactgccgtgggaatcagagaactgctgccatcaggatacgtgtgggaggggtga

SEQ ID NO: 32 - DNA, 720 bp, synthetic construct
  source 1..720: mol_type = other DNA
atggtgtcaaagggggaggagctgtttactggagtcgtgcctattctggtcgaactggacggagatgtcaacggccacaagtttagcgtgagcggcgagggggaaggagacgccacatacgggaagctgactctgaaattcatctgcaccacaggaaagctgcccgtgccttggccagccctggtcactaccctgacctacggcgtccagtgttttgctagatatccagaccacatgaagcagcatgatttctttaaatctgcaatgcccgagggatacgtgcaggaacggacaatcttctttaaggacgatggcaactataaaaccagagccgaggtgaagttcgaaggcgacacactggtcaacaggatcgagctgaaggggatcgactttaaagaagatggaaacattctgggccacaaactggagtacaactacaacagtcataaggtgtacatcactgctgataagcagaaaaacgggattaaggtcaatttcaaaacccgccacaacatcgaagacggctcagtgcagctggcagatcattaccagcagaacacacctattggcgacggccccgtgctgctgcctgataatcactatctgagcactcagtccgctctgtctaaggaccccaatgagaaacgagatcatatggtgctgctggagtttgtcaccgctgctggaatcaccctgggaatggacgagctgtataaataa

SEQ ID NO: 33 - DNA, 500 bp, synthetic construct
  source 1..500: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctggtgggtagggatgagggagggaggggcattgtgatgtaagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttc

SEQ ID NO: 34 - DNA, 710 bp, synthetic construct
  source 1..710: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgagctcgtttagtgaaccgtcagatcgcctggagacgccatccacgctgttttgacctccatagaagacaccgggaccgatccagcctccggactctag

SEQ ID NO: 35 - DNA, 57 bp, synthetic construct
  source 1..57: mol_type = other DNA
gctactaacttcagcctgctgaagcaggctggagacctggaggagaaccctgggccc

SEQ ID NO: 36 - DNA, 131 bp, synthetic construct
  source 1..131: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 37 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 38 - protein, 252 aa, Influenza A virus
MSLLTEVETYVLSIIPSGPLKAEIAQRLESVFAGKNTDLEALMEWLKTRPILSPLTKGILGFVFTLTVPSERGLQRRRFVQNALNGNGDPNNMDRAVKLYKKLKREITFHGAKEVSLSYSTGALASCMGLIYNRMGTVTTEAAFGLVCATCEQIADSQHRSHRQMATTTNPLIRHENRMVLASTTAKAMEQMAGSSEQAAEAMEVANQTRQMVHAMRTIGTHPSSSAGLKDDLLENLQAYQKRMGVQMQRFK

SEQ ID NO: 39 - protein, 232 aa, synthetic construct
MESDESGLPAMEIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVMGTGFPEDSVIFTDKIIRSNATVEHLHPMGDNDLDGSFTRTFSLRDGGYYSSVVDSHMHFKSAIHPSILQNGGPMFAFRRVEEDHSNTELGIVEYQHAFKTPDADAGEE

SEQ ID NO: 40 - DNA, 759 bp, Influenza A virus
  source 1..759: mol_type = genomic DNA
atgtctctgctcacggaggtggaaacctacgtcttgtctatcattccttcaggtcccctgaaggctgagatcgcccagcgtctcgaatcagtgttcgcaggcaaaaacaccgacctggaggctctgatggaatggctgaagactaggccaatcctgtccccgctcacaaaaggtatcctgggtttcgttttcacgttgaccgtgcctagcgagcgcggtctgcaacgccgtaggttcgttcaaaacgctctcaacggtaacggcgaccccaacaacatggatagagccgtgaagctctacaagaaactgaaacgcgagatcacgttccacggagctaaggaagtctcgctgtcctacagcactggcgctctggccagttgcatgggactcatttacaaccgcatgggtacagtgaccactgaggctgccttcggtctggtctgcgctacctgtgaacagatcgccgatagtcaacacagatcgcatcgccagatggctacaacgaccaacccactcattcgtcatgagaacaggatggttttggcttctactacagcaaaggcgatggagcagatggcaggctccagcgaacaagcagcggaggcaatggaagtcgcgaaccagaccagacaaatggttcacgcaatgcgcactatcggcacacatccgtcttcaagtgccggattgaaggacgatttgctggaaaacctccaggcttaccaaaagcgtatgggagtccagatgcaaaggttcaaatga

SEQ ID NO: 41 - DNA, 699 bp, synthetic construct
  source 1..699: mol_type = other DNA
atggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 42 - DNA, 363 bp, Antheraea pernyi
  source 1..363: mol_type = other DNA
gcaccgctaaaatggcatttgctttaccacaacgattgttctcgaaagcggggccggcgctgacgcaccgctaaaaatagcactcgctttaccagaacgattgttctcgaaagcgggtcggcgctgacgcaccgctaaaaatagcactcgctttaccagaacgattattctcgaaaaacagagccggcgctgacgcaccgctaaaaatagcactcgctttaccagaacgattgttcttgaaagaggggctggcgttgacgcccgacgatatataagcctgggcgggcgggacagagcgcactcgcagtctcgctgccgtagagtcaatatccaccgcgtcccaacacacaaacaaacgacaaa

SEQ ID NO: 43 - DNA, 382 bp, Antheraea pernyi
  source 1..382: mol_type = genomic DNA
gcaccgctaaaatggcatttgctttaccacaacgattgttctcgaaagcggggccggcgctgacgcaccgctaaaaatagcactcgctttaccagaacgattgttctcgaaagcgggtcggcgctgacgcaccgctaaaaatagcactcgctttaccagaacgattattctcgaaaaacagagccggcgctgacgcaccgctaaaaatagcactcgctttaccagaacgattgttcttgaaagaggggctggcgttgacgcccgacgtccctatcagtgatagagaatatataagctccctatcagtgatagagacgcactcgcagtctcgctgccgtagagtcaatatccaccgcgtcccaacacacaaacaaacgacaaa

SEQ ID NO: 44 - DNA, 579 bp, synthetic construct
  source 1..579: mol_type = other DNA
ggatcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgctccctatcagtgatagagactataaatactccctatcagtgatagagaaacacagttgaacagcatctgttcgaattta

SEQ ID NO: 45 - DNA, 214 bp, synthetic construct
  source 1..214: mol_type = other DNA
aaacagcaaatcgacggttttgaaatactcgtcggctctttgaccaagtaataaaatgcaagcatcaaaaatattgcaaaaatacacaaaaaaacgtaagttccttgtgcgcaataaaggccgcaagggccaccgctgtatttgtcaaaaataaacccgctatcaccccattcaacttgttgttatttttgttcattgccaacaacgtgttttg

(19) United States
(12) Patent Application Publication
(10) Pub. No.: US 2025/0242013 A1
(43) Pub. Date: Jul. 31, 2025

(54) VIRUS-LIKE PARTICLE STABLY EXPRESSED BY ANIMAL CELLS AS VACCINE ANTIGEN AGAINST COVID-19 AND INFLUENZA VIRUS

(71) Applicant: ACADEMIA SINICA, Taipei (TW)

(72) Inventors: Pei-Wen HSIAO, Taipei City (TW); Yu-Chih YANG, Taoyuan City (TW); Yi-Chun YEH, New Taipei City (TW)

(21) Appl. No.: 18/691,843

(22) PCT Filed: Sep. 14, 2022
(86) PCT No.: PCT/US22/76431

§ 371 Date: Mar. 13, 2024

Related U.S. Application Data
(60) Provisional application No. 63/244,177, filed on Sep. 14, 2021.

(51) Int. Cl. A61K 39/215; A61K 39/145; A61P 31/14; C07K 14/005; C07K 16/10; C12N 5/071; C12N 7/00; C12N 15/85

(52) U.S. Cl. CPC A61K 39/215; A61K 39/145; A61P 31/14; C07K 14/005; C07K 16/1003; C07K 16/1018; C12N 5/0686; C12N 7/00; C12N 15/85; C12N 2760/16122; C12N 2760/16123; C12N 2760/16134; C12N 2760/16151; C12N 2770/20022; C12N 2770/20023; C12N 2770/20034; C12N 2770/20051; C12N 2830/003

(57) ABSTRACT

The disclosure provides an animal cell stably expressing a virus-like particle (VLP). The disclosure also provides a method for manufacturing a virus-like particle, a virus-like particle, a vaccine composition, a method for preventing viral infection, and a method for producing antibodies.

### PRIORITY INFORMATION

This application is a 371 National Phase of International Patent Application No. PCT/US22/76431, filed Sep. 14, 2022, which claims benefit of and priority to U.S. Provisional Patent Application No. 63/244,177, filed on 14 Sep. 2021, the contents of which is incorporated by reference in its entirety.

### SEQUENCE LISTING

The instant application contains a Sequence Listing which is submitted electronically in .xml format and is hereby incorporated by reference in its entirety. The .xml copy, created on Jun. 10, 2024, is named “G4590-13800NP_20240610_UpdatedSeqListing” and is 82 kilobytes in size.

### FIELD OF THE INVENTION

This disclosure relates to vaccines. More specifically, the disclosure relates to a virus-like particle stably expressed by animal cells.

### BACKGROUND OF THE INVENTION

The use of protein as therapeutic has been approved by US-FDA and is expected to grow significantly. However, the manufacture of a protein drug depends on high-level gene expression. To produce proteins with extensive post-translational modifications, mammalian and insect cell lines appear to be the best or most acceptable expression system. Also, the capacity of producing vaccines to rapidly respond to emerging infectious diseases, such as COVID-19, pandemic H1N1 2009, etc. is vital to control outbreaks of infectious disease.

An ideal vaccine must be highly immunogenic and confer sterilizing immunity and protection from infection within safety margins. Accordingly, an advanced vaccine should comprise structure-designed antigens that display neutralizing epitopes on the surface with a stable structure, a delivery system that promotes the vaccine uptake by antigen-presenting cells, and an adjuvant activity that triggers protective immune responses through binding to immune receptors (Koff et al., Sci Transl Med 13, 2021). In addition to newly developed gene-based vaccines currently in use such as lipid nanoparticle-encapsulated messenger RNA (mRNA-LNP) and antigen-encoding adenoviral vectors, conventional virus- and protein-based vaccines are also in development (Keech et al., N Engl J Med 383, 2320-2332, 2020; Richmond et al., Lancet 397, 682-694, 2021; Kuo et al., Sci Rep 10, 20085, 2020; Krammer, Nature 586, 516-527, 2020). Due to the high risks of large-scale virus cultures and incomplete inactivation, as well as the threat of pre-existing cross-reactive immunity to previously exposed common cold coronaviruses, virus-based vaccines have not been popular in developed countries. Protein-based vaccines are generally minimally immunogenic, requiring an appropriate adjuvant to activate immune response. Virus-like particle (VLP) and nanoparticle platforms that recapitulate the particulate features of the virus and enhance delivery of antigens to lymphoid tissues and dendritic cell uptake are also promising in vaccine research.

However, it is necessary to rapidly co-express multiple genes in a cell line to enable rapid, stable, and large-scale production.

### SUMMARY OF THE INVENTION

The present disclosure relates to an expression system for rapid, stable, and large-scale production of viral vaccine.

The present disclosure provides an animal cell stably expressing a virus-like particle, comprising an inducible expression cassette for one or more site-specific recombinant VLP genes.

The animal cell may be a human cell. Examples of the animal cell include but are not limited to insect or mammalian cells. The mammalian cell may be derived from rat, mouse, guinea pig, rabbit, dog, cat, pig such as minipig, cow, horse, primates such as monkeys including a cynomolgus, or human. The insect cell may be exemplified by Lepidoptera, examples of which include but are not limited to Spodoptera frugiperda, Bombyx mori, Heliothis virescens, Heliothis zea, Mamestra brassicas, Estigmene acrea, and Trichoplusia ni.

In some embodiments of the disclosure, the virus is an enveloped virus. Examples of the enveloped virus include but are not limited to sindbis virus, rubella virus, yellow fever virus, hepatitis C virus, influenza virus, measles virus, mumps virus, human metapneumovirus, respiratory syncytial virus, vesicular stomatitis virus, rabies virus, hantaan virus, crimean-congo hemorrhagic fever virus, rift valley fever virus, coronavirus, SARS virus, LCM virus, human T-cell leukemia virus, human immune deficiency virus (HIV), marburg virus, ebola virus, human herpes viruses, vaccinia virus, and hepatitis B virus. Particularly, the virus is a coronavirus or influenza virus. In one embodiment of the disclosure, the virus is SARS-CoV-2 (COVID-19) or H5N2 or H3N2 influenza virus.

In some embodiments of the disclosure, the virus-like particle comprises a coronaviral structural protein or an influenza viral structural protein.

In one embodiment of the disclosure, the coronaviral structural protein is a structural protein of SARS-CoV-2. In some embodiments of the disclosure, the SARS-CoV-2 is Delta and Omicron variants of SARS-CoV-2. The coronaviral structural protein as disclosed herein comprises a spike protein (S), a membrane protein (M), and an envelope protein (E). Examples of the spike protein include but are not limited to a native D614G spike protein (SEQ ID NO: 6), a di-proline mutant spike protein (2P-S) (SEQ ID NO: 8), a D614G-S mutant spike protein, Delta-or spike protein (SEQ ID NO: 10), Delta-GSAS-2P spike protein (SEQ ID NO: 12), Omicron-or spike protein (SEQ ID NO: 14), Omicron-GSAS-2P spike protein (SEQ ID NO: 16). In one embodiment of the disclosure, the coronaviral structural protein is coded by a DNA sequence selected from the group consisting of SEQ ID NOs: 7, 9, 11, 13, 15, and 17.

In one embodiment of the disclosure, the influenza viral structural protein is a structural protein of H5N2 influenza virus (for example, A/duck/Taiwan/01006/2015/H5N2). The influenza viral structural protein as disclosed herein comprises a hemagglutinin (HA), neuraminidase (NA), and matrix proteins (M1 and M2). In some embodiments of the disclosure, the H5 protein is as shown in SEQ ID NO: 18, and the codon of the H5 protein is optimized as shown in SEQ ID NO: 19. In some embodiments of the disclosure, the N2 protein is as shown in SEQ ID NO: 20, and the codon of the N2 protein is optimized as shown in SEQ ID NO: 21. In one embodiment of the disclosure, the influenza viral structural protein is a structural protein of H3N2 influenza virus (for example, A/Taiwan/083/2006/H3N2). In some embodiments of the disclosure, the M1 protein is as shown in SEQ ID NO: 22, and the codon of the M1 protein is optimized as shown in SEQ ID NO: 23. In some embodiments of the disclosure, the M2 protein is as shown in SEQ ID NO: 24, and the codon of the M1 protein is optimized as shown in SEQ ID NO: 25.

In one embodiment of the disclosure, the animal cell is established by stably transfecting a target cassette comprising an inducible tetracycline-inducible promoter-reporter or a doxycycline-inducible promoter-reporter of Flp/FRT recombination system and a stably-expressed tetracycline repressor cassette; and gene swapping the target cassette and tetracycline repressor cassette through cotransfection with FLPe recombinase to accomplish site-specific insertion of all VLP genes.

In some embodiments of the disclosure, the inducible expression cassette comprises a tetracycline-inducible promoter or a doxycycline-inducible promoter. An example of the tetracycline-inducible promoter is CMV/TO promoter. Examples of the doxycycline-inducible promoter include, but are not limited to promoters of Orgyia pseudotsugata multicapsid nucleopolyhedrosis virus (OpMNPV) immediate-early 2 (IE2) and Antheraea pernyl actin-A1.

In some embodiments of the disclosure, the animal cell also stably expresses a tetracycline repressor cassette.

In some embodiments of the disclosure, the tetracycline repressor cassette comprises a tetracycline repressor gene, and a blasticidin S-resistance gene connected by a self-cleaving 2A peptide derived from porcine teschovirus-1.

In some embodiments of the disclosure, the tetracycline repressor cassette is EF1a/eIF4g-pCI-TetR-P2A-BSR cassette.

The present disclosure also provides a method for manufacturing a virus-like particle, comprising culturing the animal cell as described herein and harvesting the virus-like particle.

The present disclosure also provides a virus-like particle manufactured by the method comprising culturing the animal cell as described herein and harvesting the virus-like particle.

The present disclosure provides a vaccine composition comprising an immunologically effective amount of the virus-like particle manufactured by the method comprising culturing the animal cell as described herein and harvesting the virus-like particle.

In a further embodiment, the vaccine composition further comprises at least one adjuvant, such as Alum, squalene-based oil-in-water nano-emulsion, MF59 adjuvant, or AS03 adjuvant.

The present disclosure provides a method for preventing viral infection in a subject comprising administrating the subject the vaccine composition. The present disclosure also provides use of the vaccine composition as described herein in the manufacture of a medicament for preventing viral infection in a subject.

In some embodiments of the disclosure, the viral infection is coronaviral infection or influenza viral infection. Particularly, the viral infection is SARS-CoV-2 infection or H5N2 or H3N2 influenza viral infection.

In some embodiments of the disclosure, administration of the VLPs is for preventing viral replication or alleviating symptoms due to the viral infection.

The present disclosure also provides a method for producing antibodies, comprising administrating a subject the vaccine composition and harvest the antibodies specific to the VLPs. The present disclosure also provides use of the vaccine composition as described herein in the manufacture of a medicament for producing antibodies specific to the VLPs in a subject.

### BRIEF DESCRIPTION OF THE DRAWINGS

FIGS. 1A to 1F show production and characterization of 2P-S VLP and D614G-S VLP from 293F stable clone. FIG. 1A shows forms of the spike in 2P-S VLP and D614G-S VLP. TM, transmembrane region; CT, cytoplasmic domain. FIG. 1B shows immunofluorescence staining with anti-S1, anti-S2 antibodies, and DAPI in 293F clones stably expressing different spikes of SARS-CoV-2. FIGS. 1C and 1D show that 2P-S VLP (FIG. 1C) and D614G-S VLP (FIG. 1D) were detected by both anti-S1 and S2 antibodies in western blot. VLP samples were treated by non-reducing and non-boiling (N), non-reducing and boiling (NB), and reducing and boiling (RB) before loading. FIG. 1E shows that physical analysis of VLPs was determined by dynamic light scattering (DLS). FIG. 1F shows the central slices through the cryo-EM tomogram of 2P-S VLP and D614G-S VLP. The distribution of spikes can be observed on the surface of both VLPs. Scale bar: 100 nm.

FIGS. 2A to 2C show immune responses of VLPs in a C57BL/6 mouse. FIG. 2A shows vaccination regimen in a mouse model including immunization strategy. FIG. 2B shows that D614G-S VLP was used as antigen and detected by the anti-sera pool from mice immunized with different VLP vaccine formulas in western blot. N, non-reducing and non-boiling; NB, non-reducing and boiling; RB, reducing and boiling. FIG. 2C shows that splenocytes were collected on day 14 after boost and then stimulated without or with D614G-S VLP containing 0.75 μg of S for 24 h. The numbers of spots formed by IFN-γ and IL-4 secreting cells per 5×10⁵ splenocytes are represented. Column, geometric mean; bar, 95% confidence interval (CI).

FIGS. 3A to 3D show immunization of VLPs adjuvanted with AddaVax in hamsters. FIG. 3A shows vaccination regimen in the hamster model, including time points of immunization and viral challenge. FIG. 3B shows that neutralizing titers of anti-sera from immunized hamsters at week 2 after the booster were determined in CPE-based colorimetric live virus micro-neutralization assay. The scatter plot represents the individual data points and superimposes a horizontal line at the geometric mean with 95% CI. FIG. 3C shows that pseudovirus neutralizing titers of anti-sera from immunized hamsters were determined using pseudoviruses carrying S protein from SARS-CoV-2. Curve plot of time with individual data points; line, geometric mean; bar, 95% CI. FIG. 3D shows that D614G-S VLP could be recognized by the anti-sera pool from VLP immunized hamsters in western blot. N, non-reducing and non-boiling; NB, non-reducing and boiling.

FIGS. 4A to 4F show protective efficacy of VLP vaccines in immunized hamsters. FIG. 4A shows the medians of body weight change in immunized hamsters after challenge. Points, median; bar, 95% CI. FIGS. 4B to 4D show bodyweight change of individuals and medians after challenge in hamsters immunized with PBS (FIG. 4B), 2P-S (FIG. 4C), and D614G-S (FIG. 4D). FIG. 4E shows that the expression level of the viral E gene in the lung and duodenum of immunized hamsters at 3 dpi (left) and 6 dpi (right) was determined by RT-PCR. Plots were geometric mean with 95% CI. FIG. 4F shows that TCID₅₀ of SARS-CoV 2 in lung and duodenum tissue lysates at 3 dpi (left) and 6 dpi (right) were determined and represented as scatter plots. Points, individual data; line, geometric mean; bar, 95% CI.

FIGS. 5A to 5G show histopathological analysis of vaccinated hamsters after SARS-CoV-2 infection. FIG. 5A shows pathological analysis in H&E stain. Scale bar, 500 μm. FIG. 5B shows quantitation of consolidation level in challenged hamsters at 3 dpi and 6 dpi represented as a scatter plot. Points, individual data; line, geometric mean; bar, 95% CI. FIG. 5C shows that virus level was determined in IHC with SARS-CoV-2-N antibodies. Scale bar, 100 μm. FIGS. 5D to 5G show immune responses of challenged hamsters. Lung specimens were examined by IHC analysis using MX1 (FIG. 5D), MPO (FIG. 5E), IBA-1 (FIG. 5F), and CD3 (FIG. 5G) antibodies for IFN response, neutrophils, macrophages, and CD3 T cells, respectively.

FIGS. 6A and 6B show that 2P-S VLP (FIG. 6A) and D614G-S VLP (FIG. 6B) could be recognized by anti-sera from patients after convalescing from COVID-19 infection.

FIGS. 7A and 7B show quantitation of spike protein content in 2P-S and D614G-S VLP using anti-S1 (FIG. 7A) and anti-S2 (FIG. 7B) antibodies in western blot.

FIGS. 8A and 8B show histopathological analysis of vaccinated hamsters without infection. FIG. 8A shows H&E stain. FIG. 8B shows immune responses of challenged hamsters. Lung specimens underwent IHC analysis using MX1, MPO, IBA-1, and CD3 antibodies to examine IFN response, neutrophils, macrophages, and CD3 T cells, respectively.

FIGS. 9A to 9C show production of VLPs derived from the Delta variants of SARS-CoV-2. The repeated emergence of new SARS-CoV-2 variants with ever-increasing infectivity also suggests the fitness-enhancing mutations coming along with the accruing mutations of the spike. Therefore, we have made a series of VLP producer cell lines chasing along the up-to-date new variants of concern (VOCs). Using our VLP system in the 293F cell line, we have engineered two series of VLPs based on the spike protein sequences of the SARS-CoV-2 Delta and Omicron VOCs. We have generated clones of producer cell lines that co-express S protein carrying different mutations as marked, including the original (or-) Delta, GSAS-2P, and GSAS mutants together with M and E proteins, thus producing three different Delta-VLPs. The S protein in the VLPs was characterized by immunoblotting using anti-S1 (Cat #40592-T62, Sino Biological, Beijing, China) and anti-S2 antibodies (Cat #40590-D001, Sino Biological), and the spherical morphology of VLPs displayed respective spikes observed by electron cryo-tomography (ECT or cryoET).

FIGS. 10A to 10D show the production of VLPs derived from the Omicron variants of SARS-CoV-2. Similarly, we produced the Omicron VLPs by co-expressing Omicron S carrying different mutations as marked with M and E proteins. The S protein in the Omicron VLPs was characterized by immunoblotting using anti-S1 (Cat #40592-MM117, Sino Biological) and anti-S2 antibodies (Cat #40590-D001, Sino Biological). ETC shows the spherical morphology of the Omicron VLPs displaying respective spikes. The mean diameters of Omicron VLPs derived from S, M, and E proteins were 114.8±0.8 nm, 109.3±0.4 nm, 110.7±0.7 nm, and 96.4±0.4 nm for the original, 2P-S, RQSR-2P-S, and GSAS-2P-S mutants, respectively.

FIGS. 10E to 10H show that over-expression of S protein is sufficient to drive the release of VLPs from animal cells. Our proteomic analysis of the VLPs derived from over-expressing the ancestral S, M, and E proteins in the 293F cell line revealed that VLP incorporates almost undetectable levels of M and E proteins. Therefore, we investigated whether overexpressing S protein only may drive the budding of VLPs from the transgenic cells. Interestingly, over-expression of S protein only in the 293F cell line also produced VLPs and released in the culture medium, including the ancestral S (wild-type) and its GSGS-2P-S mutant, the Omicron S (original), and Omicron 2P-S and RQSR-S mutants (FIGS. 10E to 10G). After purification, the mean diameters of Omicron VLPs derived from Omicron S protein only were 105.5±0.4 nm, 114.1±1.0 nm, and 124.4±0.5 nm for the original, 2P-S, and RQSR-2P-S mutants, respectively (FIGS. 10E to 10G). This is the first evidence that the S protein alone of a coronavirus is sufficient to drive the budding of coronavirus-like particles (marked by arrows) within the cell and productively released the VLPs in the extracellular space and cell culture medium (FIG. 10H).

FIG. 10I shows that VLPs of the Omicron BA.1 variant can elicit neutralizing antibodies despite poor immunogenicity. The assessment of candidate vaccines has been performed by immunization using homologous prime-boost (2-shot) vaccination in mice models. The VLPs derived from the S protein of Delta and Omicron VOCs and their mutants formulated with an MF59 adjuvant (1:1, V/V, provided by RuenHuei Biopharmaceuticals Inc.) exhibited much lower immunogenicity compared with the 2P-S VLP derived from the ancestral S protein sequence. Therefore, we raised the dose of Omicron VLPs to five folds (containing 3.75 μg S protein/mouse) and compared the formulation with the ancestral 2P-S VLP (containing 0.75 μg S protein/mouse) into a divalent vaccine in the animal experiment for a new vaccine or booster vaccine. Female K18-hACE2 [B6.Cg-Tg (K18-ACE2) 2Prlmn/J] mice (n=7 or 8, 8 weeks old) were subcutaneously injected with monovalent Omicron S VLP (original BA.1 or its RQSR-2P mutant), and their combinations with the ancestral 2P-S VLP (1:5 W/W) admixed with AS03 adjuvant (1:1, V/V, provided by RuenHuei Biopharmaceuticals Inc.) as respective divalent vaccines on day 0 and 21 and bled on day 35. Specific IgG titers in the pooled antisera against all vaccines containing the Omicron VLPs reached GMT: 2×10⁵ in ELISA. The neutralization potency of immune sera against Omicron BA.1 pseudotyped lentiviruses indicated by 50% pseudovirus neutralization titers (PVNT₅₀) of O-RQSR-2P-S and the two divalent vaccines were significantly higher than the O-or-S (Omicron original spike). The PVNT₅₀ values (neutralization titers) against BA.5 of mice immunized by the divalent vaccines (2P-S plus either Omicron O-or-S or O-RQSR-2P-S) were significantly higher than the BA.1-based monovalent groups, suggesting that neutralizing antibody response generated by Omicron VLP was rather specific to the same Omicron subvariant, unlike the ancestral 2P-S VLP vaccine. Our data suggest the dampening immunogenicity of Omicron VLP requires an increment of antigen dose (containing 3.75 μg S protein/mouse) and formulation with more potent adjuvants, such as AS03 or others, to augment and prolong the cellular and humoral immune responses provoked by the VLP vaccine targeting Omicron and future VOCs.

The data have demonstrated that VLP displaying Omicron S with RQSR-2P mutations can be effective to comprise strain-matched vaccine.

FIGS. 11A to 11H show creation of stable 293F cell line producing H5N2-VLP and characterization of H5N2-VLP. FIG. 11A in step I shows that human 293F cell line was stably transfected with the GFP reporter plasmid to trap a highly expressing genome locus. Single-cell clones were isolated and characterized for clones with a single-copy reporter as founder cell lines. In step II, a funder cell line was then transfected with FLPe and the donor plasmid to swap GFP with the H5N2-VLPs gene cluster. FIG. 11B shows that gene-swapped cells were enriched based on loss-of-GFP after doxycycline induction. FIG. 11C shows that single-cell clones were isolated and characterized for their inducible expression of VLP genes. FIG. 11D shows that VLP-producer cells were scaled up in suspension culture and induced by adding 1 μg/mL doxycycline in the medium. H5N2-VLP were harvested from the conditioned medium and purified by sucrose density gradient ultracentrifugation. Purified H5N2-VLP (batch-1 and batch-2) were analyzed for their hemagglutination activity. FIG. 11E shows that purified H5N2-VLP was negative stained by 2% uranyl acetate and observed by transmission electron microscopy (TEM) at 100,000× magnification. FIG. 11F shows that the particle size and distributions of purified H5N2-VLP was characterized by dynamic light scattering (DLS). FIG. 11G shows that NA activity was measured by NA-star Influenza Neuraminidase Inhibitor Resistance Detection Kit (Thermo Fisher). Per μg H5N2-VLPs provided 150,000 RLU/sec of NA activity. FIG. 11H shows that HA protein of purified H5N2-VLPs was analyzed by western blotting using an anti-H5 antibody.

FIGS. 12A to 12E show creation of stable insect High-Five cell line producing H5N2-VLP and characterization of H5N2-VLP. FIG. 12A shows that insect High-5 cell line was stably transfected with the GFP reporter plasmid to trap a highly expressing genome locus. A funder cell line was then transfected with insect promoter-driven FLPe and the donor plasmid to swap GFP with the H5N2-VLPs gene cluster. Gene-swapped cells were enriched based on loss-of-GFP after doxycycline induction. FIG. 12B shows that single-cell clones were isolated and characterized for clones with a single-copy reporter as founder cell lines. GFP image of a representative founder cell line. FIG. 12C shows that single-cell clones were isolated and characterized for their inducible expression of VLP genes by qRT-PCR. FIG. 12D shows that neuraminidase activity of VLP-producer cells were measured after scaled up in suspension culture and induced by adding 1 μg/mL doxycycline in the medium and measured by NA-Star Influenza Neuraminidase Inhibitor Resistance Detection Kit (Thermo Fisher). FIG. 12E shows that urified H5N2-VLP was negative stained by 2% uranyl acetate and observed by transmission electron microscopy (TEM) at 200,000× magnification.

FIG. 13 shows the target cassette for a mammalian cell system.

FIG. 14 shows the target cassette for an insect cell system.

FIG. 15 shows the map of pGEMT-RMCE1-CMV to -sfGFP.

FIG. 16 shows the map of pUC57.Insect RMCR1.

### DETAILED DESCRIPTION OF THE INVENTION

The present invention can be more readily understood by reference to the following detailed description of various embodiments of the invention, the examples, and the chemical drawings and tables with their relevant descriptions. It is also to be understood that the terminology used herein is for the purpose of describing particular embodiments only and is not intended to be limiting.

As utilized in accordance with the present disclosure, the following terms, unless otherwise indicated, shall be understood to have the following meaning:

As used herein, the use of “or” means “and/or” unless stated otherwise. In the context of a multiple dependent claim, the use of “or” refers back to more than one preceding independent or dependent claim in the alternative only.

It must be noted that, as used in the specification and the appended claims, the singular forms “a,” “an” and “the” include plural referents unless the context clearly dictates otherwise. Thus, unless otherwise required by context, singular terms shall include the plural and plural terms shall include the singular.

As used herein, the term “optional” or “optionally” means that the subsequently described event or circumstance may or may not occur, and that the description includes instances where the said event or circumstance occurs and instances where it does not. For example, the phrase “optionally comprising an agent” means that the agent may or may not exist.

The expressions “stable expression” and “stably expressing” as used herein are intended to mean that the genetic material that is being stably expressed and/or is integrated permanently and stably in the genome of the host cell, and thus has the same expression potential over time as the native genetic material of the host cell.

As used herein, the term “virus-like particle” refers to a structure resembling a virus particle. Moreover, a virus-like particle in accordance with the disclosure is non-replicative and noninfectious since it lacks all or part of the viral genome, in particular the replicative and infectious components of the viral genome. A virus-like particle in accordance with the disclosure may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages have a spherical form of icosahedral symmetry. The term “capsid-like structure” as used herein, refers to a macromolecular assembly composed of viral protein subunits reassembling the capsid morphology in the previously defined sense but deviating from the typical symmetrical assembly while maintaining a sufficient degree of order and repetitiveness.

In one embodiment of the disclosure, the virus-like particle is a multimer of viral structural proteins, preferably of viral coat proteins and/or viral envelope proteins that does not comprise polynucleotides but which otherwise has properties of a virus, e.g. binds to cell surface receptors, is internalized with the receptor, is stable in blood, and/or comprises glycoproteins etc.

The term “viral structural proteins” is used in the context of the present disclosure to refer to viral coat proteins or viral envelope glycoproteins.

The term “cell line” as used herein refers to cultured cells that can be passed (divided) more than once. The disclosure relates to cell lines that can be passed more than 2 times, up to 200 times, or more and includes any integer therebetween.

The term “transfection” refers to the introduction of nucleic acid molecules, such as DNA or RNA (e.g. mRNA) molecules, into cells, preferably into animal cells. In the context of the present disclosure, the term “transfection” encompasses any method known in the art for introducing nucleic acid molecules into cells, preferably into animal cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethyleneimine and others. Preferably, the introduction is non-viral.

As used herein, the term “expression cassette” refers to a recombinant DNA molecule containing a desired coding sequence and appropriate nucleic acid sequences necessary for the expression of the operably linked coding sequence in a particular host organism. Eukaryotic cells are known to utilize promoters, enhancers, and termination and polyadenylation signals.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

The term “subject” as used herein denotes any animal, preferably a mammal, and more preferably a human. Examples of subjects include humans, non-human primates, rodents, guinea pigs, rabbits, sheep, pigs, goats, cows, horses, dogs and cats.

As used herein, the term “immunologically effective amount” refers to an amount of a composition sufficient to induce an immune response in an individual when introduced into that subject. The amount of a composition necessary to be immunologically effective varies according many factors including to the composition, the presence of other components in the composition (e.g. adjuvants), the antigen, the route of immunization, the subject, the prior immune or physiologic state etc.

The term “adjuvant” as used herein refers to non-specific stimulators of the immune response or substances that allow generation of a depot in the host which when combined with the composition of the invention provides for an even more enhanced and/or prolonged immune response, preferably cytokine production. A variety of adjuvants is known in the art and useful in the invention. Preferred adjuvants are selected from the group consisting of incomplete Freund's adjuvant, aluminum containing adjuvants, modified muramyl dipeptide, surface active substances such as lysolecithin, pluronic polyols, polyanions, peptides, oil emulsions, keyhole limpet hemocyanins, dinitrophenol, BCG (bacille Calmette Guerin) Corynebacterium parvum, ligands of toll-like receptors (TLR) which include but are not limited to peptidoglycans, lipopolysaccharides and their derivatives, poly I:C, immunostimulatory oligonucleotides, imidazoquinolines such as resiquimod and imiquimod, flagellins, monophosphoryl lipid immunomodulator, AdjuVax 100a, QS-21, QS-18, GPI-0100, CRL1005, MF-59, OM-174, OM-197, OM-294, Virosomal adjuvant technology and any mixture thereof. A highly preferred adjuvant for the purpose of the invention is aluminium containing adjuvant, preferably an aluminium containing mineral gel, most preferably alhydrogel. In a highly preferred embodiment said adjuvant is alhydrogel. The term adjuvant also encompasses a mixture of any of the substances listed above. Particles of the invention, preferably VLPs, have been generally described as adjuvant. However, the term “adjuvant”, as used within the context of this application, refers to an adjuvant not being the particle of the disclosure, in particular not the VLP used for the compositions. In each case, the term adjuvant refers to an adjuvant used in addition to the said particle.

As used herein, the term “immune response” refers to any action by the immune system of an individual that is directed against a molecule or compound, such as an antigen. In mammals, the immune response includes both the activities of cells and the production of soluble molecules such as cytokines and antibodies. The term thus includes a humoral immune response and/or cellular immune response leading to the activation or proliferation of B- and/or T-lymphocytes. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent.

Virus-like particles have recently emerged as promising and versatile tools to confront the virus infection. Recombinant VLPs produced in cell culture systems contain no viral genome and cannot replicate. For vaccine purposes, VLPs are an efficacious and safer alternative to the live-attenuated virus and whole-inactivated virus.

Accordingly, the present disclosure aims to provide an animal cell stably expressing a virus-like particle. The present disclosure not only significantly advances the quality of manufacturer's master cell bank and provides the highest speed-to-market and expression-levels, but also avoids unintended drawbacks due to the time-consuming selection process of random insertion of transfected DNA into the genome of cGMP banked master cell lines. In some embodiments of the disclosure, stable clones of animal cell lines that harbor the designed transgenes with features of gene insertion at the same locus as predetermined and exhibiting a maximized level of gene expression are preferred to generate producer cell lines for the manufacture of a medicament for producing antibodies and preventing viral infection in a subject.

The present disclosure provides a process to create a stable cell line that co-express multiple genes, which are of great use to produce vaccine candidates. For achieving a more satisfactory immune response, the animal cell is utilized in the disclosure for stably expressing the VLP. To make the expression cell line highly productive and swiftly generated by site-specific chromosomal gene insertion, the mammalian and insect cell lines are provided to co-express multiple viral genes and efficiently produce VLPs. In some embodiments of the disclosure, the VLPs mimic coronavirus SARS-CoV-2 and avian influenza virus H5N2 or H3N2.

The VLPs comprise four major coronaviral structural proteins, including the spike (S) protein, membrane (M) protein, and the envelope (E) protein, which are regarded as promising vaccine candidates and useful diagnostics to detect human antibodies after natural infection by SARS-CoV-2. Concomitant expression of S, M, and E proteins of coronavirus in the same cell of higher animals can initiate the self-assembly and release of VLPs.

The viral entry of SARS-CoV-2 is mediated by the surface glycoprotein spike (S) that binds angiotensin-converting enzyme 2 (ACE2) of host cells and undergoes a prefusion to postfusion conformational change (Walls et al., Cell 176, 1026-1039 e1015, 2019). Although receptor binding domain (RBD) of S is well-recognized to provide immunogenicity in vaccine development, other neutralizing antibodies targeting the S1 domain outside the RBD and S2 domain of SARS-CoV-2 S have also been identified in the blood of convalescent patients (Brouwer et al., Science 369, 643-650, 2020; Huang et al., PLoS Pathog 17, e1009352, 2021). S proteins with 2P mutations situated at the turn between the central helix and heptad repeat 1 of S2 have been constructed to stabilize the S protein in its prefusion structure and avoid the shedding of the S1 subunit in COVID-19 vaccines (Tostanoski et al., Nat Med 26, 1694-1700, 2020; Koff et al., Sci Transl Med 13, 2021; Corbett et al., N Engl J Med 383, 1544-1555, 2020; Polack et al., N Engl J Med 383, 2603-2615, 2020; Voysey et al., Lancet 397, 99-111, 2021).

In some embodiments of the disclosure, using a site-specific stable transfection system, two enveloped VLPs displaying the full-length trimmers of uncleavable 2P-S and D614G-S are provided and produced by the suspension culture of the human 293F cell line as immunogen candidates. The S protein of VLP 2P-S carries 5 mutations (3 disabling the furin-type S1/S2 cleavage and 2 stabilizing prolines) and D614G-S VLP displays the native form S of the D614G variant.

Examples of the spike protein include but are not limited to a native D614G spike protein (SEQ ID NO: 6), a di-proline mutant spike protein (2P-S) (SEQ ID NO: 8), a D614G-S mutant spike protein, Delta-or spike protein (SEQ ID NO: 10), Delta-GSAS 2P spike protein (SEQ ID NO: 12), Omicron-or spike protein (SEQ ID NO: 14), Omicron-GSAS 2P spike protein (SEQ ID NO: 16). In one embodiment of the disclosure, the codon of the native D614G spike protein is optimized as shown in SEQ ID NO: 7; the codon of the di-proline mutant spike protein (2P-S) is optimized as shown in SEQ ID NO: 9; the codon of the Delta-or spike protein is optimized as shown in SEQ ID NO: 11; the codon of the Delta-GSAS 2P spike protein is optimized as shown in SEQ ID NO: 13; the codon of the Omicron-or spike protein is optimized as shown in SEQ ID NO: 15; the codon of the Omicron-GSAS 2P spike protein is optimized as shown in SEQ ID NO: 17.

In some embodiments of the disclosure, the VLPs comprise hemagglutinin (HA), neuraminidase (NA), and matrix proteins (M1 and M2) of influenza viruses. Co-expressing the structural proteins in the same animal cell leads to self-assembly and release of VLPs exhibiting the native structure of entire virions and has proven useful in eliciting neutralizing antibodies, thus representing a promising vaccine candidate and native antigens to develop diagnostics.

In some embodiments of the disclosure, the influenza virus is H5N2, such as A/duck/Taiwan/01006/2015/H5N2. The influenza viral structural protein as disclosed herein comprises hemagglutinin (HA), neuraminidase (NA), and matrix proteins (M1 and M2). In some embodiments of the disclosure, the H5 protein is as shown in SEQ ID NO: 18, and the codon of the H5 protein is optimized as shown in SEQ ID NO: 19. In some embodiments of the disclosure, the N2 protein is as shown in SEQ ID NO: 20, and the codon of the N2 protein is optimized as shown in SEQ ID NO: 21.

In one embodiment of the disclosure, the influenza virus is H5N2, such as A/Taiwan/083/2006/H3N2. In some embodiments of the disclosure, the M1 protein is as shown in SEQ ID NO: 22, and the codon of the M1 protein is optimized as shown in SEQ ID NO: 23. In some embodiments of the disclosure, the M2 protein is as shown in SEQ ID NO: 24, and the codon of the M1 protein is optimized as shown in SEQ ID NO: 25.

In some embodiments of the disclosure, the animal cell is manufactured by a “swift multi-gene overexpression system” comprising (1) trapping of a chromosomal locus that allows the highest-level and inducible expression of a GFP gene in a host cell line to obtain the founder cell lines, (2) engineering of the donor plasmid that harbors a cluster of multiple transgenes individually driven by a CMV/TO promoter, and (3) “swapping the trapped GFP gene” with the donor gene cluster through FLPe recombinase-mediated cassette exchange (RMCE) to accomplish site-specific insertion of all transgene.

Particularly, the animal cell as described herein is manufactured by (1) trapping of a chromosomal locus that allows the highest level of inducible foreign gene expression in mammal and insect cell lines, (2) engineering of the donor plasmid harboring an FRT-flanked cluster of several transgene cassettes, CMV/TO promoter-driven, and expressing 3 or 4 viral structural proteins, (3) “gene swapping” the donor cassettes through cotransfection with FLPe recombinase to accomplish site-specific insertion of all transgenes, and (4) producing and purifying VLPs.

In one embodiment of the disclosure, the inducible expression cassette comprises a tetracycline-inducible promoter as a target cassette such as a CMV/TO cassette of Flp/FRT recombination system, flanked by an F- and an F3 site. In some embodiments of the disclosure, the animal cell also stably expresses a tetracycline repressor cassette. In some embodiments of the disclosure, the stably expressed tetracycline repressor cassette comprises a tetracycline repressor gene, and a blasticidin S-resistance gene connected by a self-cleaving 2A peptide derived from porcine teschovirus-1. In some embodiments of the disclosure, the stably expression cassette is EF1a/eIF4g-pCI-TetR-P2A-BSR cassette.

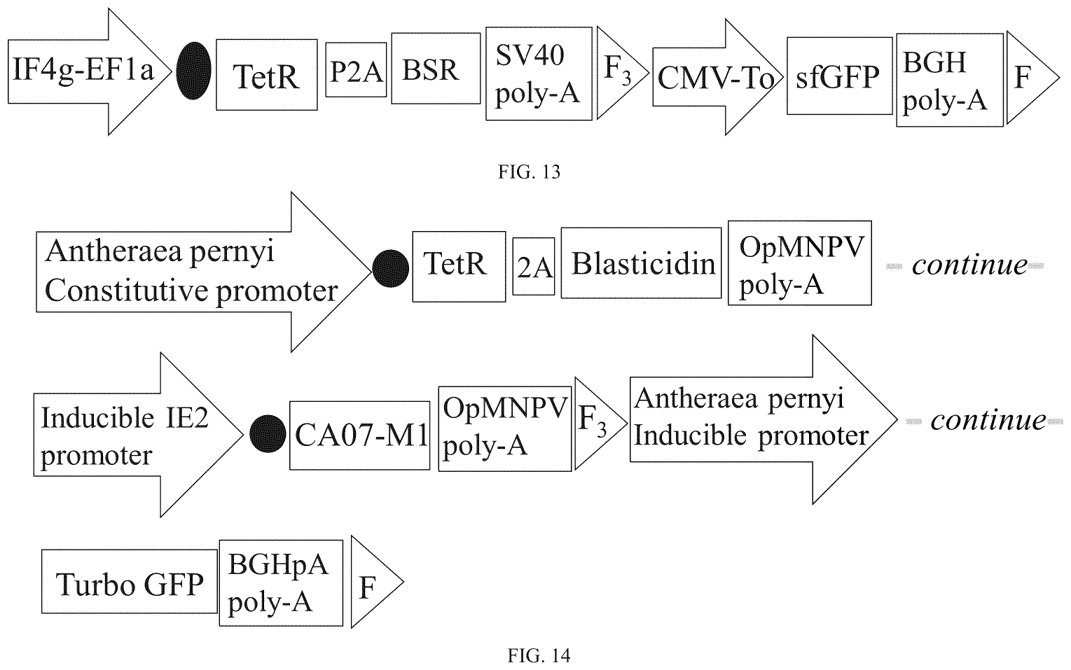

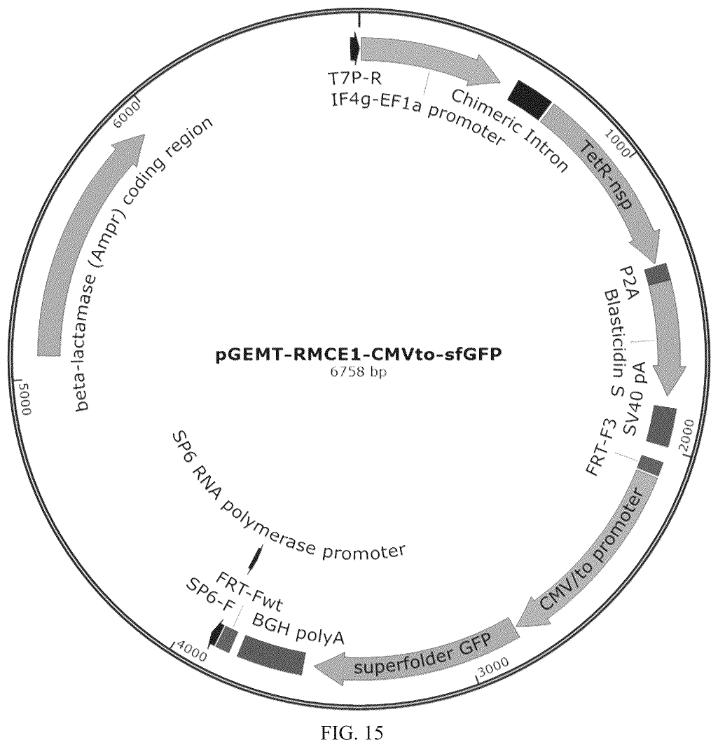

In one embodiment of the disclosure, the target cassette for a mammalian cell system is shown in FIG. 13, and the map of the plasmid pGEMT-RMCE1-CMV to -sfGFP is shown in FIG. 15.

In one embodiment of the disclosure, the inducible expression cassette comprises a doxycycline-inducible promoter. Examples of the doxycycline-inducible promoter include, but are not limited to promoters of Orgyia pseudotsugata multicapsid nucleopolyhedrosis virus (OpMNPV) immediate-early 2 (IE2) and Antheraea pernyl actin-A1. In principle, constitutively expressed Tet-repressor holds back the expression from IE2 and A. pernyl actin-A1 promoters through binding to the tet operator 2 (TetO2) inserted downstream of the TATA-box until the Tet-repressor is unleashed by doxycycline treatment. The inducible expression cassette further comprises CMV/TO driven VLP genes, distributed into 2 or 3 tandem genes. The entire cluster of transgenes is flanked by two FRT sites (F and Fn) to be the target cassette of FLPe flippase. The donor plasmid and FLPe expression plasmid were cotransfected into founder cell lines. The cells were sorted again by loss-of-reporter and gain-of-HA expression in the VLP genes.

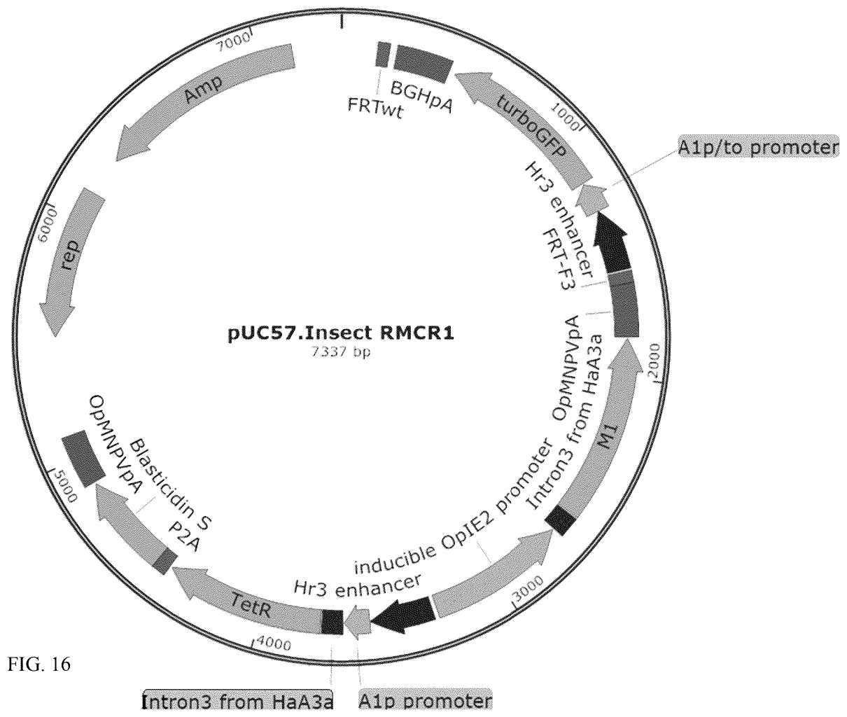

In one embodiment of the disclosure, the target cassette for an insect cell system is shown in FIG. 14, and the map of the plasmid pUC57.Insect RMCR1 is shown in FIG. 16.

The present disclosure also provides a method for manufacturing a virus-like particle, comprising culturing the animal cell and harvesting the virus-like particle.

In some embodiments of the disclosure, the method comprises culturing of the animal cell, removing cell debris and other large aggregates, and purifying the VLPs by a two-step sucrose-gradient (30% and then 40%-60%) ultracentrifugation or 2-step column chromatography using Crapto Q and Capto Core 700 multimodal (MMC) and diafiltration, or 2-step column chromatography using Capto De VirS and Capto Core 700.

The present disclosure also provides a virus-like particle manufactured by the method comprising culturing the animal cell as described herein and harvesting the virus-like particle.

The present disclosure provides a vaccine composition comprising an immunologically effective amount of the virus-like particle manufactured by the method comprising culturing the animal cell as described herein and harvesting the virus-like particle.

In a further embodiment, the vaccine composition further comprises at least one adjuvant, such as Alum or incomplete Freund's adjuvant.

The present disclosure provides a method for preventing viral infection in a subject comprising administrating the subject the vaccine composition. The present disclosure also provides use of the vaccine composition as described herein in the manufacture of a medicament for preventing viral infection in a subject.

In some embodiments of the disclosure, the viral infection is a coronaviral infection or influenza viral infection. Particularly, the viral infection is SARS-CoV-2 infection or H5N2 or H3N2 influenza viral infection.

In some embodiments of the disclosure, administration of the VLPs is for preventing viral replication or alleviating symptoms due to the viral infection. An example of the symptoms due to the SARS-CoV-2 infection includes, but is not limited to, pneumonia.

The present disclosure also provides a method for producing antibodies, comprising administrating a subject the vaccine composition and harvesting the antibodies specific to the VLPs. The present disclosure also provides use of the vaccine composition as described herein in the manufacture of a medicament for producing antibodies specific to the VLPs in a subject.

Particularly, the antibodies are neutralizing antibodies. Furthermore, the neutralizing antibodies may be applied in treating the viral infection and the symptoms due to the viral infection.

The following examples are provided to aid those skilled in the art in practicing the present invention.

### EXAMPLES

### Materials and Methods

### Inducible Expression Cassette

To construct an inducible expression cassette, we adapt the tetracycline-inducible promoter (CMV/TO) obtained from pcDNA4/TO (Invitrogen) linked to a generic chimeric intron obtained from pCI (Promega, Madison, WI), a recombinant open-reading frame, and a poly-adenylation signal from bovine growth hormone gene (BGH polyA) or SV40 polyA. To halt the CMV/TO promoter until the induction of tetracycline or doxycycline, we introduce a constitutive expression of Tet repressor (TetR, from pcDNA6/TR and optimized for mammalian codon usage). To ensure TetR expression in stably transfected cells, TetR is designed to co-repress with the blasticidin S-resistance gene (bsr, the Borrelia coriaceae cytidine deaminase) connected by a self-cleaving 2A peptide derived from porcine teschovirus-1 (P2A) for negative selection. Using CMV/TO-GFP as a reporter conjugated to the EF1a/IF4g-pCI-TR-P2A-BSR gene (pUC57.Insect RMCR1 or pGEMT-RMCE1-CMV to -sfGFP) we have generated funder cell clones by random insertion in the host cell genome and selected the clones exhibiting a maximized Tet-inducible expression of GFP. During our optimization of the green fluorescence in different host cell lines, superfolder GFP was used in mammalian cell lines and turbo GFP was used in insect cell lines. Both GFP and the BSR used were optimized for mammalian codon usage.

The sequence of Tet repressor is shown in SEQ ID NO: 26 with optimized codon of SEQ ID NO: 30; the sequence of BSR is shown in SEQ ID NO: 27 with optimized codon of SEQ ID NO: 31; the sequence of sfGFP is shown in SEQ ID NO: 28 with optimized codon of SEQ ID NO: 32; the sequence of P2A is shown in SEQ ID NO: 29 with optimized codon of SEQ ID NO: 35; the sequence of hEF1α/eIF4γ promoter is shown in SEQ ID NO: 33; the sequence of CVM/TO is shown in SEQ ID NO: 34; the sequence of polyadenylation signals of SV40 is shown in SEQ ID NO: 36; the sequence of polyadenylation signals of bovine growth hormone (BGH) is shown in SEQ ID NO: 37;

The sequence of M1 protein from A/California/07/2009/H1N1 is shown in SEQ ID NO: 40; the sequence of turboGFP is shown in SEQ ID NO: 41; the sequence of constitutive Antheraea pernyi promoter is shown in SEQ ID NO: 42; the sequence of inducible Antheraea pernyi promoter is shown in SEQ ID NO: 43; the sequence of inducible IE2 is shown in SEQ ID NO: 44; the sequence of Polyadenylation signal of OpMNPV is shown in SEQ ID NO: 45.

### Tetracycline Repressor Cassette

The constitutive expression cassette was not used in the viral genes. Instead, it is for the expression of tet-repressor to halt the CMV/TO promoter until the induction of tetracycline or doxycycline.

### Establishment of Expression Cells

VLP expression cells were established in FreeStyle 293F cells. 293F founder cells were established by stable transfection with an inducible expression of CMV/TO-GFP as a target cassette of Flp/FRT recombination system, flanked by an F- and an F3 site, and constitutive expression of EF1a/eIF4g-TetR-P2A-BSD cassette. Double transfected cells were negatively selected by blasticidin S and clonally isolated by GFP intensity. For site-specific recombination of S/M/E genes, founder cells were co-transfected with a donor plasmid carrying a CMV/TO-S-CMV-TO-M-IRES-E expression cassette flanked by an F- and an F3 site and a plasmid expressing FLPe recombinase, and then isolated clones lost GFP and gained S/M/E genes.

### Immunofluorescence staining

VLP-expression cells were induced with Dox or vehicle control for 48 h. The cells were then fixed in 4% paraformaldehyde for 10 min and immersed in 0.05% Triton X-100 for 1 min. The cells were then blocked with 3% BSA, incubated with specific primary antibodies, washed, followed by incubation with goat anti-rabbit or goat anti-mouse IgG conjugated with Cy2 or Cy3 dye. Fluorescence images were captured by inverted fluorescence microscopy (Observer D1, Zeiss). The antibodies used in this study were S1 (40150-R007, Sino Biological), and S2 (GTX632604, Genetex).

### Cell Suspension Culture and VLP Production

The 293F VLP producer cells (about 2×10⁶/mL) were seeded in FreeStyle 293 expression medium (Gibco) in 2 L Erlenmeyer flasks, and suspension-cultured by agitation at 150 rpm in a humidified incubator at 37° C. with 5% CO₂. To induce the VLP expression and secretion from cells, the culture medium was added with 1 μg/ml doxycycline for 72 h, conditioned medium thereof was harvested, filtered with 0.45 μm Stericap, concentrated by Vivaflow 50 (Sartorius Stedim Biotech), and chromatographically purified using Capto Q and Capto Core 700 columns (GE Healthcare, Swenson) mounted on an ÄKTA pure 25 system (Cytiva, GE Healthcare, Swenson) at 4° C.

### Protein Assay and Western Blotting

The protein constituents of purified VLPs were quantified by Quant-iT Protein Assay Kit (Invitrogen). Purified VLPs were mixed with Lämmle SDS-PAGE sample buffer, without reducing agent and boiling (N), without reducing agent but boiled for 2 min (NB), with reducing agent and boiled (RB), followed by SDS-PAGE in a 4-12% gradient gel and then subjected to western blot analyses.

### Dynamic Light Scattering (DLS) Determination of Particle Size

VLP samples were diluted to 0.1 μg/mL in 20 mM phosphate buffer, pH 7.4, passed through the 0.45-μm filter, and analyzed on a Nano ZS particle-size analyzer (Malvern Zetasizer, Malvern Instruments). Each sample was measured by DLS for 60 sec, consecutively twice. The size distribution that integrates the number of particles in each size class was converted from the intensity-based measurement using the accompanying software (Nanov510) and represented as a diagram of curves showing the frequency distribution of the sample where the area under the curve was proportional to the numbers of particles detected in the indicated size range. The average diameters of VLPs were then calculated as the mean size of particle population±standard deviation (SD) of three independent experiments.

### Cryo-EM

To prepare a cryo-EM grid, an aliquot (approx. 4 μL) of purified VLPs was added to the glow-discharged Quantifoil R2/2 holey carbon grid (Quatifoil GmbH, Germany). The grid was blotted with filter paper on both sides for 3 s and then plunge frozen into liquid ethane cooled by liquid nitrogen using a Thermo Scientific Vitrobot System (Mark IV). The cryo-EM grid was stored in liquid nitrogen until imaging, with all subsequent steps performed below −160° C. to prevent devitrification.

Cryo-EM grids were clipped, mounted in a cassette, transferred with a nano-cap, and loaded into a 200 kV Talos Arctica transmission electron microscope (Thermo Fisher Scientific). The alignment was performed in the nanoprobe mode (spot size 3, gun lens 4 with C2 lens set to 43.8%) to achieve parallel beam alignment and coma-free alignment. Cryo-EM images were recorded with a Falcon III detector (Thermo Fisher Scientific) operating in linear mode at a magnification of 92,000× with a pixel size of 1.1 Å/pixel. The defocus used for imaging was set to approx. 2.5 μm and the dose rate was set to approx. 20 e−/Å2 per second to yield a total dose of approx. 50 e−/Å2 in 2.5 s. EPU-2.2.0 software (Thermo Fisher Scientific) was used to collect cryo-EM images.

Cryo-EM grids were loaded into a 200 kV Talos Arctica transmission electron microscope (Thermo Fisher Scientific). A Falcon III detector (Thermo Fisher Scientific) was used to record the tilt images at a magnification of 73,000× with a pixel size of 1.4 Å/pixel. The defocus for imaging was set to approx. 6 μm. Tomography-4.10.0 software (Thermo Fisher Scientific) was used to collect the tomographic tilt images with a tilt range of ±60° and a constant angular increment of 3° (from +20° to −60°, and then from +20° to +60°). A dose of approx. 3 e−/Å2 was used for each tilt, resulting in a total dose of approx. 120 e−/Å2 among 41 tilt images. The 3D tomogram was reconstructed from the tilt images by Inspect3D-4.2 software (Thermo Fisher Scientific). The tilted images were aligned, the tilt axis was adjusted, and tomographic reconstruction was performed with simultaneous iterative reconstruction technique (SIRT). The tomographic reconstruction was visualized with the “Inspect Stack” function in Inspect3D-4.2 software (Thermo Fisher Scientific).

### Immunization Strategy

Eight-week-old female C57BL/6 mice and male golden Syrian hamsters were purchased from the National Laboratory Animal Center, Academia Sinica, Taiwan. VLP formula was adjuvanted with or without Alhydrogel or AddaVax adjuvant (InvivoGen) in a 1:1 mixture. Mice and hamsters aged 9 weeks were immunized twice by subcutaneous injection and the prime-boost interval time was 10 days (Liang et al., Sci Transl Med 9, 2017). The injection volumes were 100 μL in mice and 200 μL in hamsters. Blood samples were collected before and every week after the booster shot.

### Determination of the Specificity of ELISA

ELISA plates (Nunc) were coated with D614G-S VLPs at 4° C. overnight and blocked with StartingBlock blocking buffer (Thermo Fisher Scientific). Hamster serum samples of indicated dilution were added into coated ELISA plates and then incubated at 37° C. for 1 h, traced with HRP-conjugated secondary Ab, and developed with TMB substrate (Pierce), finally absorbance was measured at 450 nm (Power Wave XS, Bio-Tek). Three washes with PBST buffer were applied between each ELISA step. IgG subtype in hamster serum was detected with respective secondary antibodies, anti-hamster IgG1 (1940-05, SouthernBiotech) and anti-hamster IgG2 and IgG3 (1935-05, SouthernBiotech).

### ELISpot Assay

IFN-γ or IL-4 in mice were determined using an ELISpot assay kit (R&D Systems). Immunized mice were euthanized by carbon dioxide in the second week post booster. Splenocytes were harvested and seeded 5×10⁵/well in 96-well plates, pre-coated with anti-IFN-γ or anti-IL-4 Ab for 24 h. After three washes with buffer and incubation with detection antibody at 2-8° C. overnight, plates were washed three times with buffer and incubated with streptavidin-AP at room temperature for 1 h. They were then washed again three times, and then the color was developed by incubation with BCIP/NBT chromogen at room temperature for 1 h, and the plates were rinsed with deionized water.

### Pseudovirus Neutralization Assay

293T/17-ACE2 cells and pseudovirus were provided by the National RNAi Core Facility (Academia Sinica, Taiwan). 293T/17-ACE2 cells were seeded in 96 well plates with 1% heat-inactivated FBS DMEM media. Immune sera were diluted 2-fold from 1:20. An equal volume of pseudovirus was incubated with diluted immune sera at 37° C. for 1 h. Pseudoviruses carried the S protein from SARS-CoV-2 and the luciferase gene as a reporter. After incubation, the sera-pseudovirus mixture was added to cells and centrifuged at 1,100×g for 30 min and the cells were cultured in a humidified incubator at 37° C. with 5% CO₂ for 24 h. The culture media was refreshed with 1% FBS DMEM medium and incubated for another 48 h. Luciferase activity was measured with Firefly Luciferase Assay Kit (Biotium).

### CPE Neutralization Assay

Serially diluted antibodies were incubated with 100 TCID₅₀ SARS-CoV-2 TCDC #4 (hCoV-19/Taiwan/4/2020) at 37° C. for 1 h. The mixtures were then added to pre-seeded Vero E6 cells and cultured for 4 days. Cells were fixed with 10% formaldehyde and stained with 0.5% crystal violet for 20 min. The plates were washed with tap water and scored for infection. The 50% protective titer was calculated by Reed and Muench method.

### Viral Challenge Experiments

Viral challenge infection experiments in the hamster model were approved by the Institutional Animal Care and Use Committee (IACUC) and the P-3 Lab of Academia Sinica. Vaccinated Syrian hamsters were anesthetized and intranasally challenged with 1×10⁵ PFU of SARS-CoV-2 TCDC #4 (hCoV-19/Taiwan/4/2020, GISAID accession ID: EPI_ISL_411927) (lot: IBMS20200819, 8.0×10⁵ PFU/mL in a volume of 125 μL. All hamsters were weighed daily after the SARS-CoV-2 challenge infection. Surviving hamsters from the infection experiments were euthanized using carbon dioxide.

### Quantification of Viral Titer in Tissue by Cell Culture Infectious Assay

Hamster tissue was homogenized in 600 μL of DMEM with 2% FBS and 1% penicillin/streptomycin using a homogenizer. Tissue homogenate was centrifuged at 15,000 rpm for 5 min and the supernatant was collected for live virus titration. Briefly, 10-fold serial dilutions of each sample were added onto Vero E6 cell monolayer in quadruplicate and grown for 4 days. Cells were then fixed with 10% formaldehyde and stained with 0.5% crystal violet for 20 min. The plates were washed with tap water and scored for infection. The 50% tissue culture infectious dose (TCID₅₀)/mL was calculated by the Reed and Muench method.

### Real-Time RT-PCR for SARS-CoV-2 RNA Quantification

To measure the RNA levels of SARS-CoV-2, specific primers targeting the 26,141 to 26,253 region of the envelope (E) gene of the SARS-CoV-2 genome were used by the TaqMan real-time RT-PCR method described in the previous study (Corman et al., Euro Surveill 25, 2020). Forward primer E-Sarbeco-F1 (5′-ACAGGTACGTTAATAGTTAATAGCGT-3′, SEQ ID NO. 1) and the reverse primer E-Sarbeco-R2 (5′-ATATTGCAGCAGTACGCACACA-3′, SEQ ID NO. 2), in addition to the probe E-Sarbeco-P1 (5′-FAM-ACACTAGCCATCCTTACTGCGCTTCG (SEQ ID NO. 3)-BBQ-3′) were used. A total of 30 μL RNA solution was collected from each sample using RNeasy Mini Kit (QIAGEN) according to the manufacturer's instructions. A 5 μL aliquot of RNA was added into a total 25 μL mixture of the Superscript III one-step RT-PCR system with Platinum Taq Polymerase (Thermo Fisher Scientific). The final reaction mix contained 400 nM forward and reverse primers, 200 nM probe, 1.6 mM of deoxy-ribonucleoside triphosphate (dNTP), 4 mM magnesium sulfate, 50 nM ROX reference dye, and 1 μL of the enzyme mixture. Cycling conditions were performed using a one-step PCR protocol: 55° C. for 10 min for first-strand cDNA synthesis, followed by 3 min at 94° C. and 45 amplification cycles at 94° C. for 15 s and 58° C. for 30 s. Data was collected and calculated by Applied Biosystems 7500 Real-Time PCR System (Thermo Fisher Scientific). A synthetic 113-bp oligonucleotide fragment was used as a qPCR standard to calibrate copy numbers of the viral genome. The oligonucleotides were synthesized by Genomics BioSci and Tech (Taipei, Taiwan).

### Histopathological Analysis and IHC

Tissues were fixed in 4% paraformaldehyde for 3 days, transferred to PBS, paraffin-embedded within 7 days, and block sectioned at 4 μm. Slides were baked at 37° C. overnight, then deparaffinized in xylene and rehydrated through a series of graded ethanol to distilled water. Histopathological analysis was performed by hematoxylin and eosin stain (Muto Pure Chemicals) and then bluing using Tacha's bluing solution (Biocare). For SARS-CoV-2-N IHC, antigen retrieval was performed using a pressure cooker for 15 min at 90° C. in Reveal Decloaker buffer (Biocare). For CD3, MX1, IBA1, and MPO IHC, antigen retrieval was performed using a pressure cooker for 10 min at 85° C. in Target retrieval solution pH 9.0 (Dako). Slides were washed with PBS and then treated with 3% hydrogen peroxide for 10 min. Slides were washed with PBS and blocking was performed with a background buster (Innovex) for 1 h. Primary antibody anti-SARS-CoV2 N (40588-T62, Sino Biological, at 1:500), anti-CD3 (ab16669, Abcam, at 1:50), anti-MX1 (13750-1-AP, Proteintech, at 1:100), anti-IBA1 antibody (10904-1-AP, Proteintech, at 1:500) or anti-MPO (ab208670, Abcam, at 1:500) was incubated at 4° C. overnight. Multivalent secondary antibody (Innovex) was applied for 15 min and treated by peroxidase enzyme (Innovex) for 15 min. Betazoid DAB Chromogen kit was used and then counterstained with hematoxylin followed by bluing using Tacha's bluing solution (Biocare).

### Statistical Analysis

The analysis was performed using GraphPad Prism 7.05 (GraphPad Software). Comparison of data between groups in ELISpot, body weight, viral RNA, TCID₅₀, and consolidation was performed using two-way analysis of variance (ANOVA). CPE and pseudovirus neutralizing titers were assessed by one-way ANOVA. Neutralization data were log₂ transformed. Viral RNA and TCID₅₀ were log₁₀ transformed. P values of less than 0.05 were considered significant.

### Example 1 Production of COVID-19 VLP in Mammalian Cells

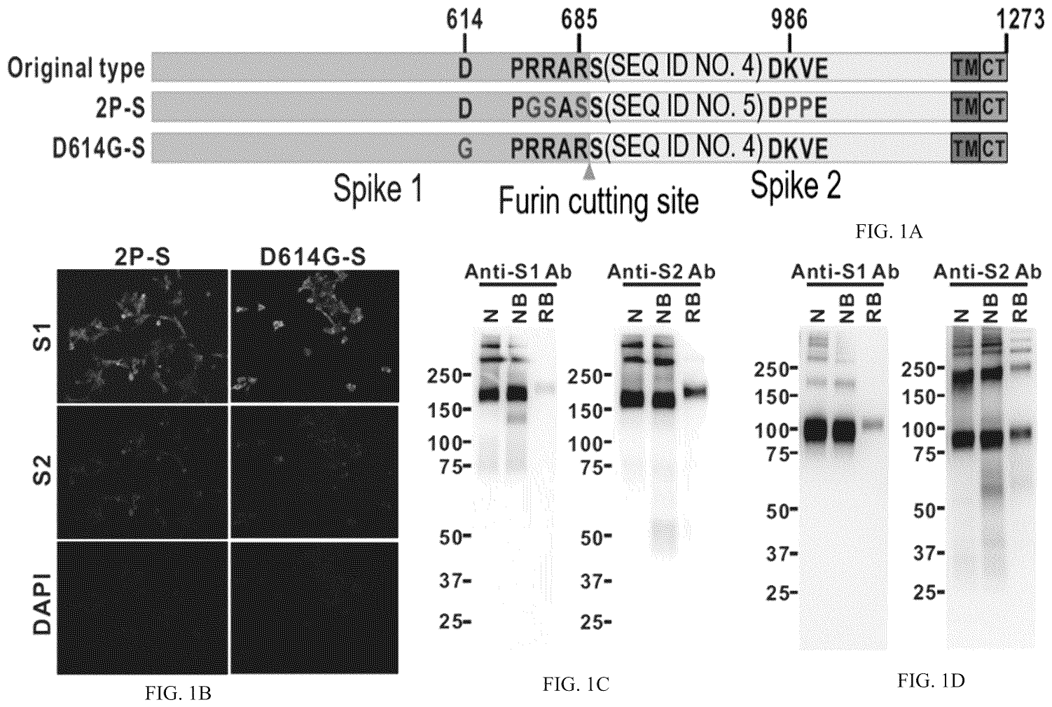

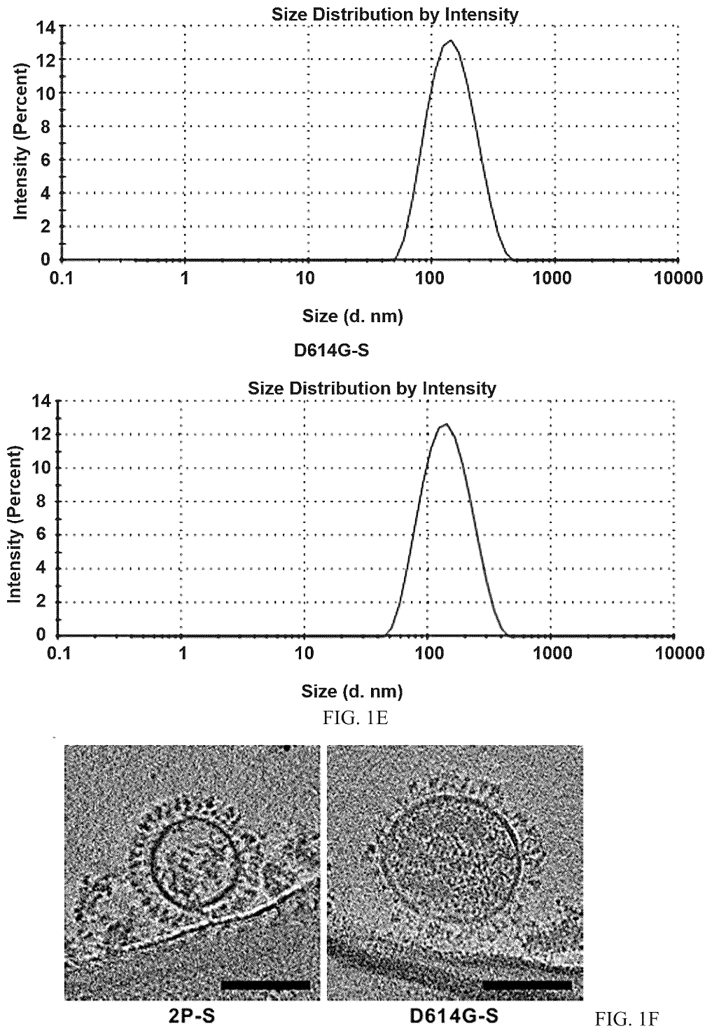

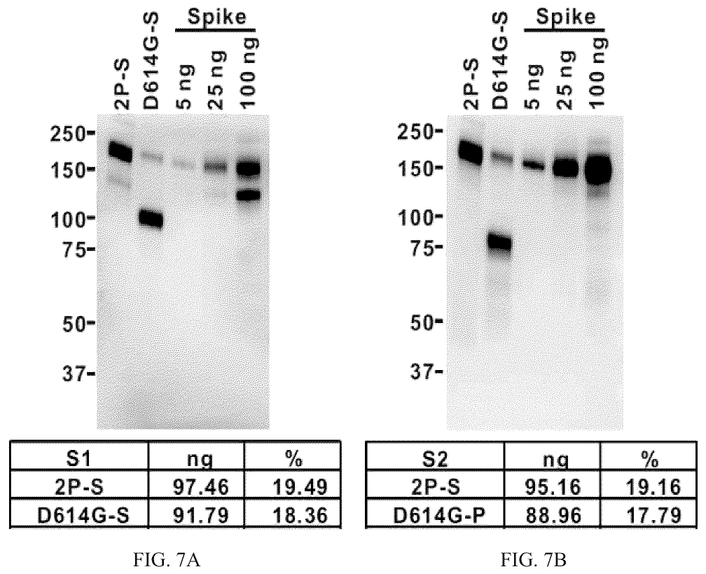

To efficiently produce VLP antigens on a large scale, 293F stable clones were established by site-specifically inserting a gene cluster in the genome to coexpress the spike (S), membrane (M), and envelop (E) proteins of SARS-CoV-2. S protein sequences encoding a prefusion stabilized 2P-S (SEQ ID NO: 8) and the native D614G variant S (SEQ ID NO: 6) were constructed as antigen candidates for the VLP vaccines (FIG. 1A). Using our platform, VLPs from 293F stable clones achieved high yields from 25 to 31 mg/L. The expression of S proteins in the producer cells was detected by immunofluorescent assay (IFA) using anti-S1 and anti-S2 antibodies (FIG. 1B). And, the S proteins of the VLPs were analyzed by western blotting using specific antibodies after purification from the conditioned medium (FIGS. 1C and 1D). As designed, the S protein was either full-length S (approx. 180K) in 2P-S or proteolytically cleaved into S1 (100K) and S2 (85K) in D614G-S, with some oligomers detected at molecular weights higher than 250K. Both VLPs (2P-S and D614G-S) could be recognized by commercial anti-S1 and anti-S2 and human antibodies from the plasma of convalescent COVID-19 patients. Of note, denaturation of S protein by treating VLPs with a reducing agent and boiling disrupted the S protein structure and diminished the recognition of antibodies, more significantly by anti-S1 than anti-S2 (FIGS. 1C and 1D, and FIGS. 6A and 6B). The content of S protein in each VLP was quantified as approximately 20% of the total VLP protein (averagely 19.3% in 2P-S, and 18.0% in D614G-S) by summation of the S protein signals (separately detected by S1 and S2 monoclonal antibodies) and interpolating from a standard curve of recombinant S-2P protein (FIGS. 7A and 7B). Using dynamic light scattering (DLS) analysis, the average particle sizes of 2P-S and D614G-S were 127.2 and 123.9 nm, respectively (FIG. 1E). As analyzed by cryogenic electron microscopy (cryo-EM), the morphology of both 2P-S and D614G-S was characterized by a clear corona of spikes (FIG. 1F). While the D614G-S VLP displayed flexible spikes likely due to the conformational pliability of the three hinges (hip, knee, and ankle) lately identified within the stalk (S2) domain tilting at different angles, the 2P-S displayed uniformly bristling spikes (Turonova et al., Science 370, 203-208, 2020). These results suggested 2P-S VLP and D614G-S VLP, respectively, presenting the full-length prefusion form of spikes protruding upright versus bending at varying degrees and directions.

### Example 2 VLPs Induced Th1 and Th2 Responses in a Mouse Model

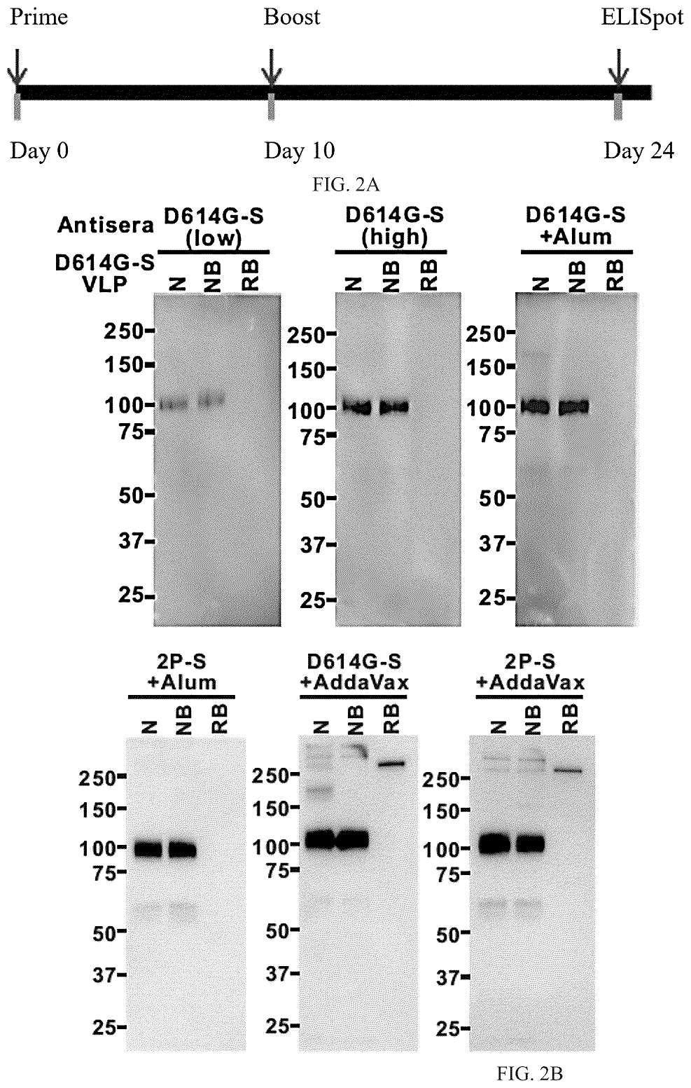

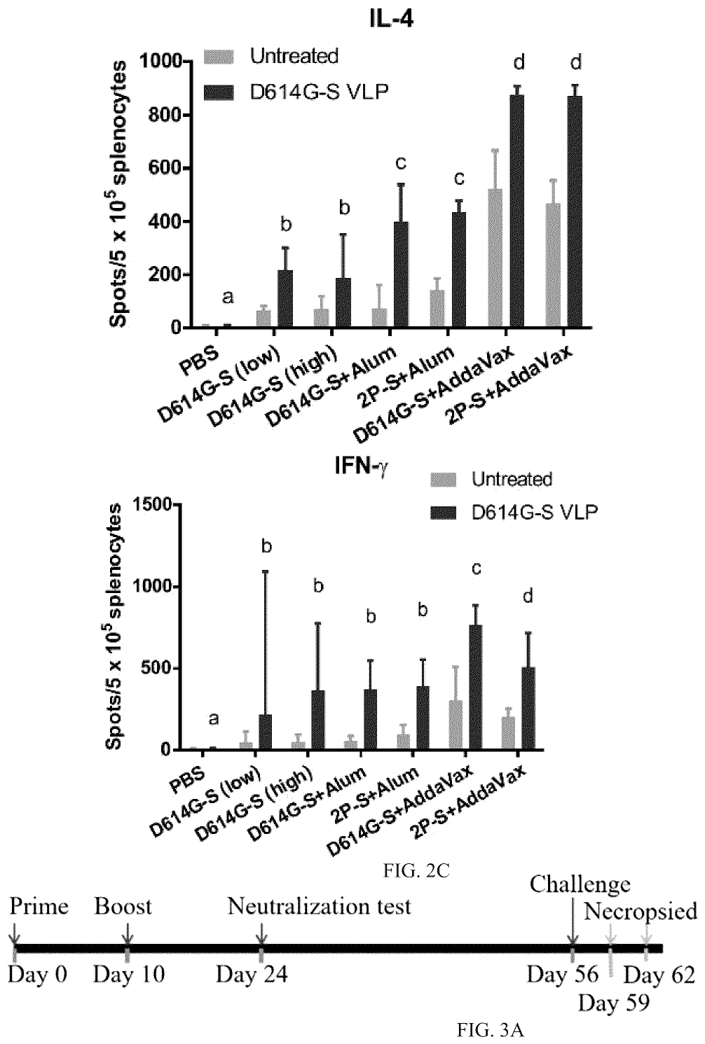

The elicitation of balanced Th1/Th2 responses by vaccination is a necessary criterion in the development of a COVID-19 vaccine to avoid the possibility of VAERD induced by Th2-biased responses after vaccination (Acosta et al., Clin Vaccine Immunol 23, 189-195, 2015; Bottazzi et al., Microbes Infect 22, 403-404, 2020). To assess the potentials of these two VLPs and identify a favorable adjuvant for the VLP formula to intensify the immune response, a homologous prime-boost vaccination strategy was performed, identifying the immunogenicity in the C57BL/6 mouse model (FIG. 2A). We immunized the mice with D614G-S VLP alone containing 0.75 μg (low) or 2.25 μg (high) of S protein, and VLPs containing 0.75 μg of S protein and adjuvanted with aluminum hydroxide gel (alum) or AddaVax. We used D614G-S VLP as a diagnostic antigen because of its resemblance to the ancestral wild-type SARS-CoV-2. In ELISA assay, VLPs adjuvanted with AddaVax induced high antibody titers of approximately 2×10⁵ similar to the alum group, which were higher than the unadjuvanted VLP (5×10⁴ and 1×10⁵, respectively, at the high and low doses of antigens) (Table 1). Similar effects were also seen in the western blot analysis using pooled antisera of the same groups, where stronger signals were seen in the AddaVax and alum groups (FIG. 2B). Furthermore, cell-mediated immune responses were analyzed by ELISpot assay. Interestingly, D614G-S VLP alone could prime both Th1 and Th2 responses, and only AddaVax further boosted the IFNγ (Th1) responses. While both alum and AddaVax augmented the IL-4 (Th2) response, the adjuvant effect of AddaVax on IL-4 was also higher than alum (FIG. 2C). These findings suggested that VLPs primed both Th1 and Th2 responses and AddaVax was a more effective adjuvant than alum to formulate with the VLP vaccine.

TABLE 1
ELISA titers after vaccination

| Species | Group | Total IgG | IgG1 | IgG2 + IgG3 |
|---|---|---|---|---|
| C57BL/6 | D614G-S-low | 5 × 10⁴ | | |
| | D614G-S-high | 1 × 10⁵ | | |
| | D614G-S + Alum | 2 × 10⁵ | | |
| | 2P-S + Alum | 2 × 10⁵ | | |
| | D614G-S + AddaVax | 2 × 10⁵ | | |
| | 2P-S + AddaVax | 2 × 10⁵ | | |
| Hamster | D614G-S + AddaVax | 2.4 × 10⁵ | 2 × 10⁵ | 5 × 10⁴ |
| | 2P-S + AddaVax | 2.4 × 10⁵ | 2 × 10⁵ | 7.5 × 10⁴ |

### Example 3 VLPs Adjuvanted With AddaVax Induced Neutralizing Antibodies in the Hamster Model

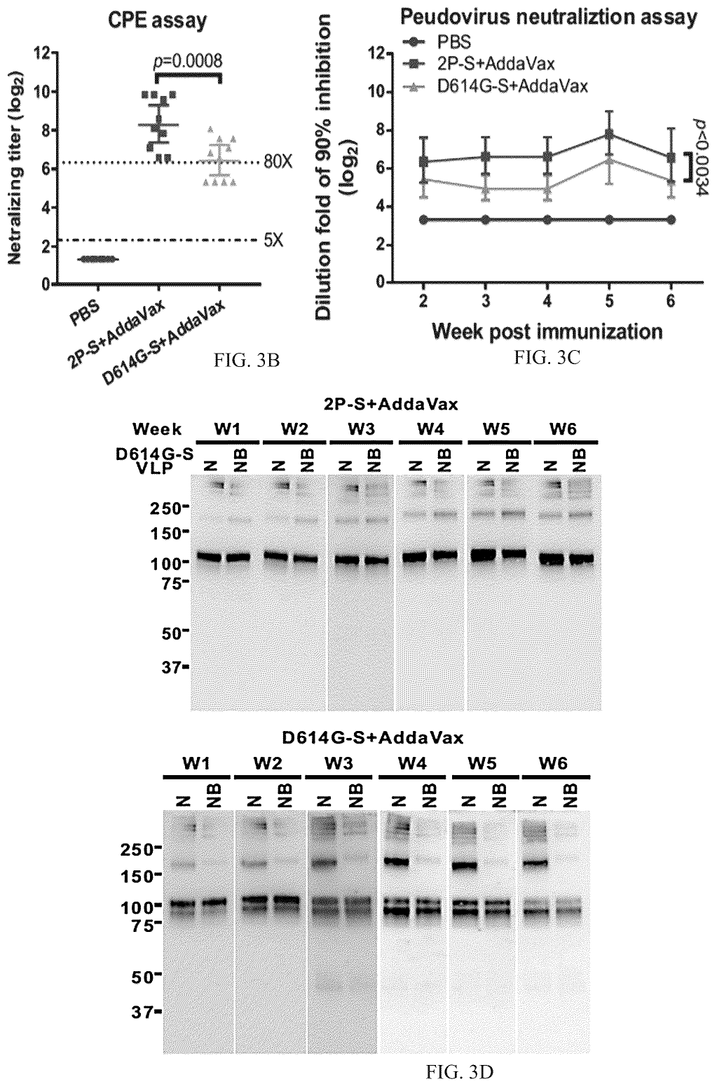

To identify the protective efficiency of the VLP-based vaccines, we then immunized golden Syrian hamsters with AddaVax-adjuvanted VLPs at a dose equivalent to 1.5 μg of S protein. The regimen of immunization and challenge is shown in FIG. 3A. Two weeks after the prime-boost immunization, both vaccines elicited high IgG titers in serum, about 2.4×10⁵ fold in ELISA assay (Table 1). Further diagnosis of the IgG subclasses induced by the VLP vaccines showed that antisera from these two vaccine groups comprised higher titers (2×10⁵) of IgG1 (Th2 response) than the IgG2+IgG3 (Th1 response) titers that were 7.5×10⁴ and 5×10⁴, respectively, in the 2P-S and D614G-S groups (Table 1). The NAb titers of antisera bled 2 weeks after the booster vaccination were initially analyzed against the cytopathic effect (CPE) observed in Vero E6 cell infection with SARS-CoV-2. The NAb titers in the 2P-S group were approximately 468, which is a 4-fold higher titer compared with the D614G-S group; 4 hamsters out of 10 in the D614G-S group had NAb titers lower than 80 (FIG. 3B). The longitudinal NAb titers were further analyzed by the S-pseudovirus neutralization assay in relevance to the viral S protein binding to the host hACE2 receptor. The NAb titers of 2P-S needed to inhibit 90% S-pseudovirus infection were higher than those of the D614G-S and mock groups from 2 weeks after the booster, and the NAb titers remained high for at least 6 weeks after the booster (FIG. 3C). In a parallel examination using western blot analyses, the anti-sera from the 2P-S group predominantly recognized the S1 subunit within the D614G-S VLP, which mimics the viral envelope. In contrast, the D614G-S anti-sera recognized both S1 and S2 one week after the booster vaccination, but the major recognition signal shifted from S1 to S2 three weeks after the booster (FIG. 3D). These results indicated that 2P-S adjuvanted with AddaVax was able to spark a higher-titer NAb and stable recognition toward S1 compared to the D614G-S vaccine which could change the epitope targets of antibodies.

### Example 4 VLPs Adjuvanted With AddaVax Prevented the Viral Replication and COVID-19 Symptoms After Challenge in the Hamster Model

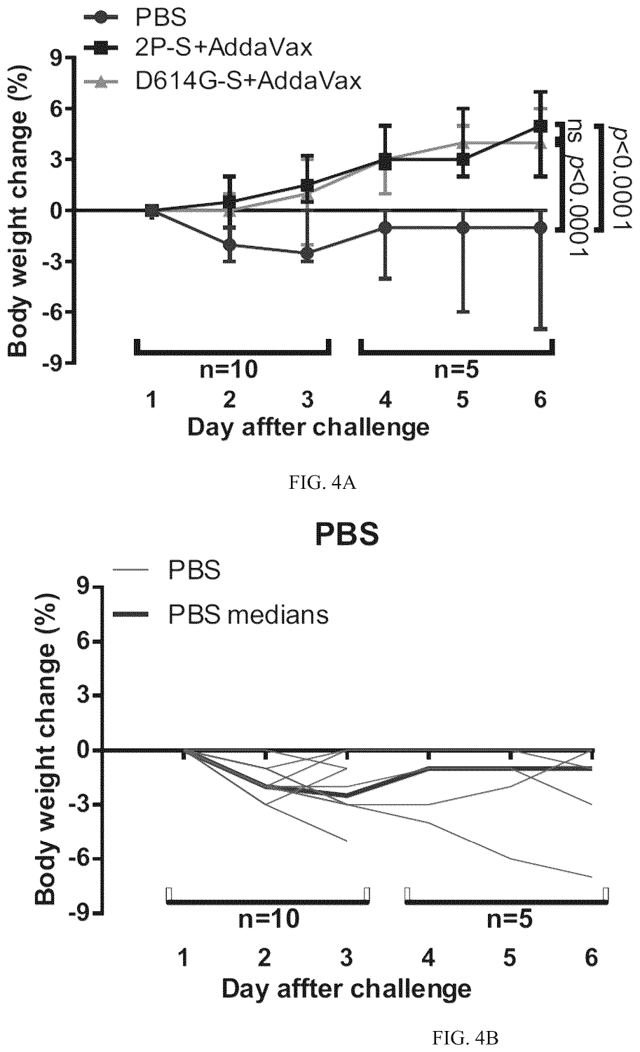

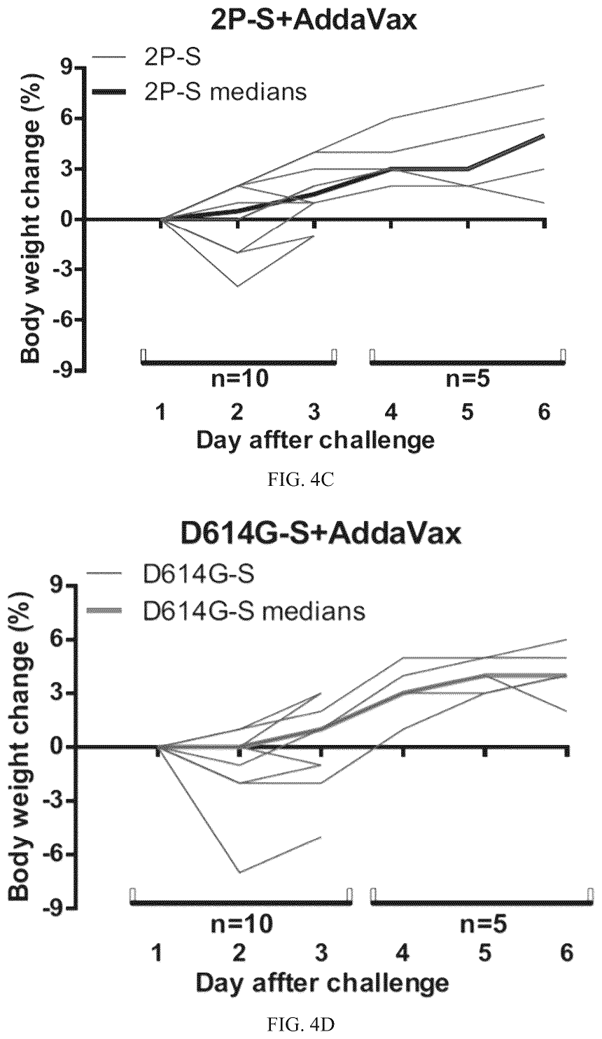

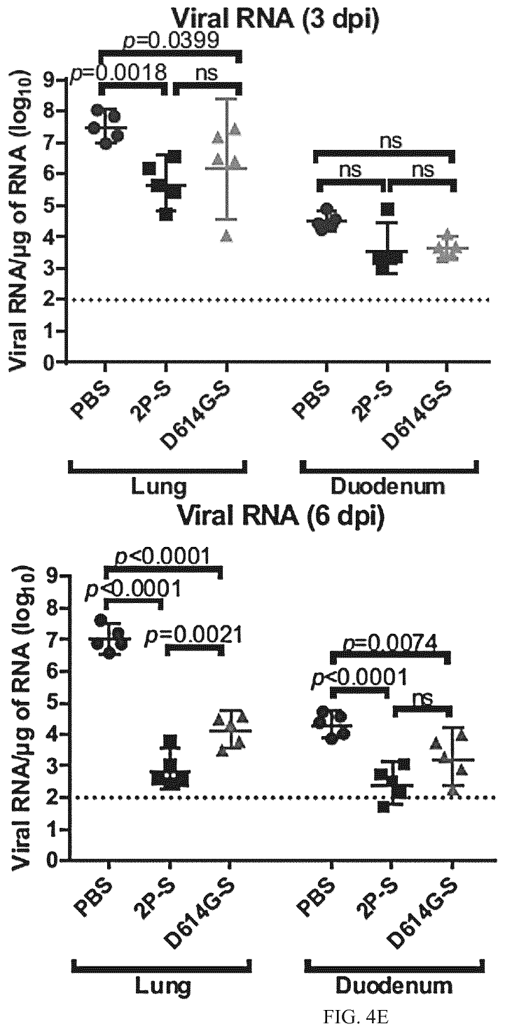

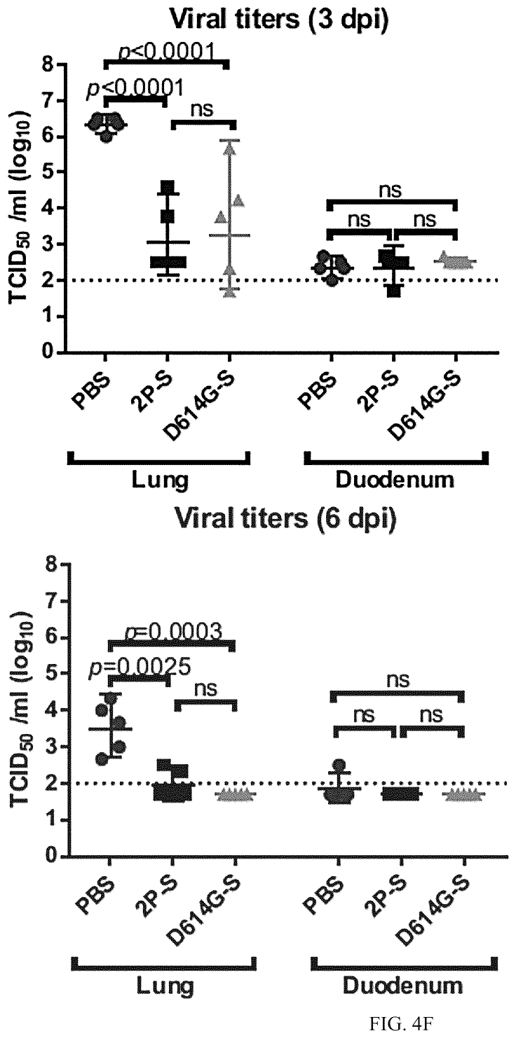

To assess the prophylactic efficacy of the VLP vaccine, hamsters immunized with PBS (mock), 2P-S, and D614G-S adjuvanted with AddaVax were challenged by intranasal inoculation with 1×10⁵ plaque-forming units (PFU) of SARS-CoV-2 at 46 days post-booster vaccination and necropsied at 3 days post-infection (3 dpi) and 6 dpi (FIG. 4A). The viral infection led to significant body weight loss in all the mock (PBS) vaccinated hamsters. In contrast, the majority of hamsters inoculated with the 2P-S vaccine and half of the hamsters inoculated with the D614G-S vaccine gained body weight beginning from 2 days after infection (FIGS. 4A to 4D). At 3 dpi and 6 dpi, the viral replication, inflammation, and pathology were diagnosed in the lung and duodenum based on the viral RNA (vRNA, E gene) and replication-competent virus (tissue culture infectious dose, TCID₅₀) of SARS-CoV-2. Both vaccines reduced the vRNA in the lungs at 3 dpi. Although 2P-S appeared more potent than D614G-S in curbing the lung vRNA synthesis at 3 dpi, the difference did not reach statistical significance due to the large variation in D614G-S (FIG. 4E). At 6 dpi, the vRNA levels of 2P-S lungs were only 0.01% of the mock levels and close to the detection limit and the D614G-S lungs were 0.1% of the mock levels (FIG. 4E). The vRNA in the duodenum was low, and both vaccines were similarly effective. Compared to the high TCID₅₀ in the mock lung (2.3×10⁶ at 3 dpi and 7.5×10³ at 6 dpi), both 2P-S and D614G-S substantially restrained the lung TCID₅₀ at various levels at 3 dpi and almost all detected below or near 100-fold (minimal dilution) at 6 dpi, whereas the viral titers in the duodenum in all groups were too low to detect any TCID₅₀ reduction upon vaccination (FIG. 4F). These results indicated the AddaVax-adjuvanted 2P-S and D614G-S both effectively reduced the viral replication and infection of SARS-CoV-2.

### Example 5 VLP Vaccines Protected Hamsters From COVID-19 Pneumonia

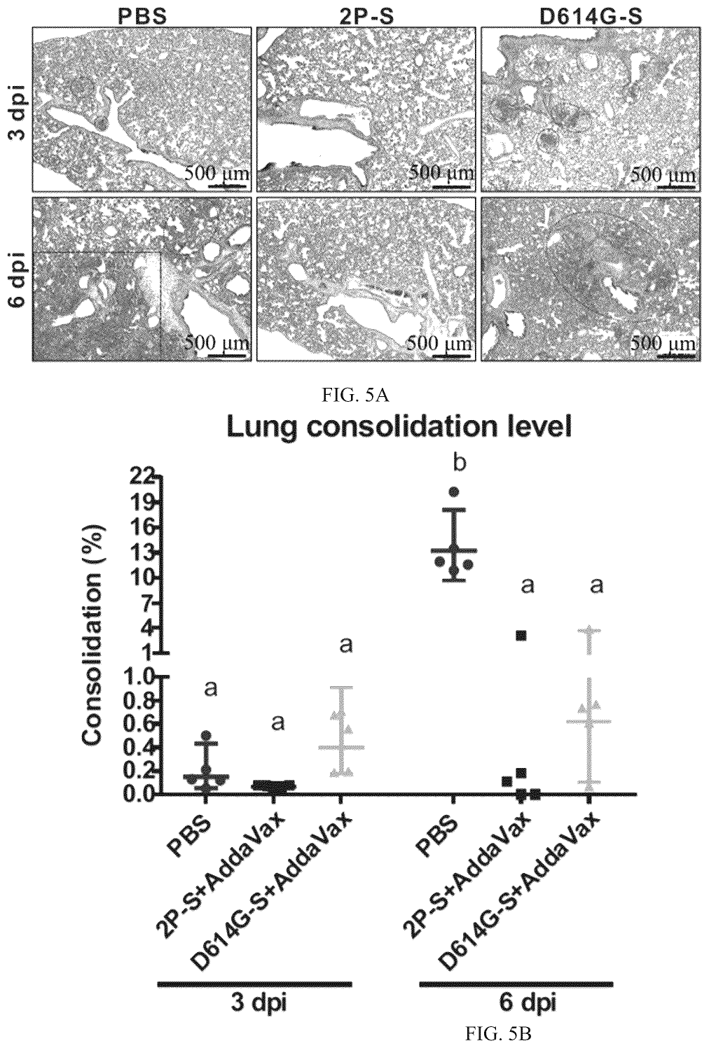

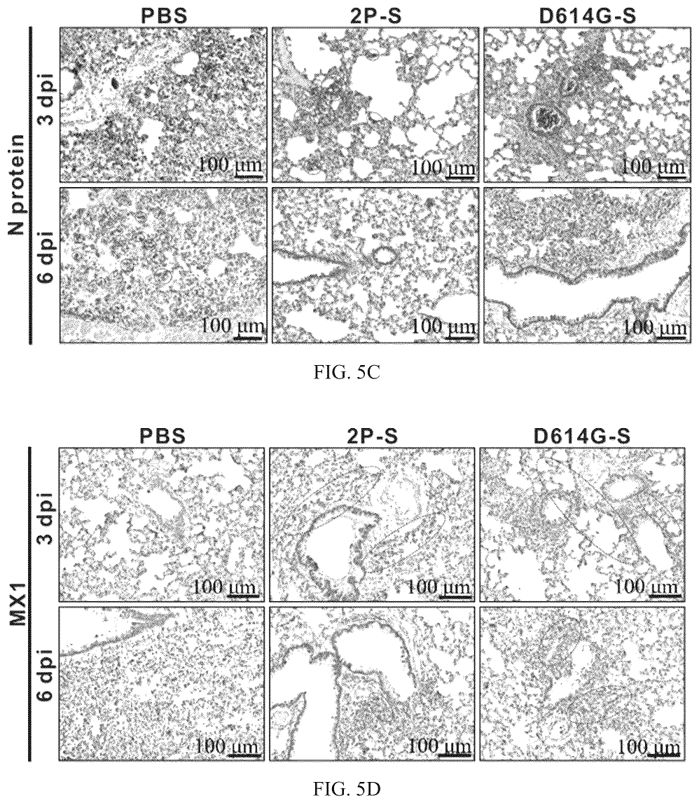

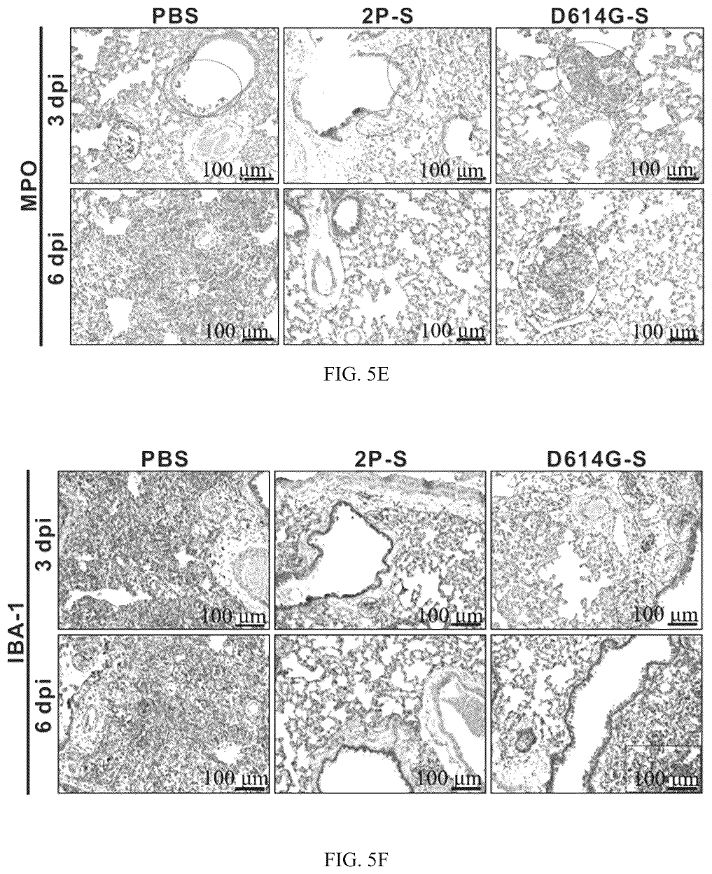

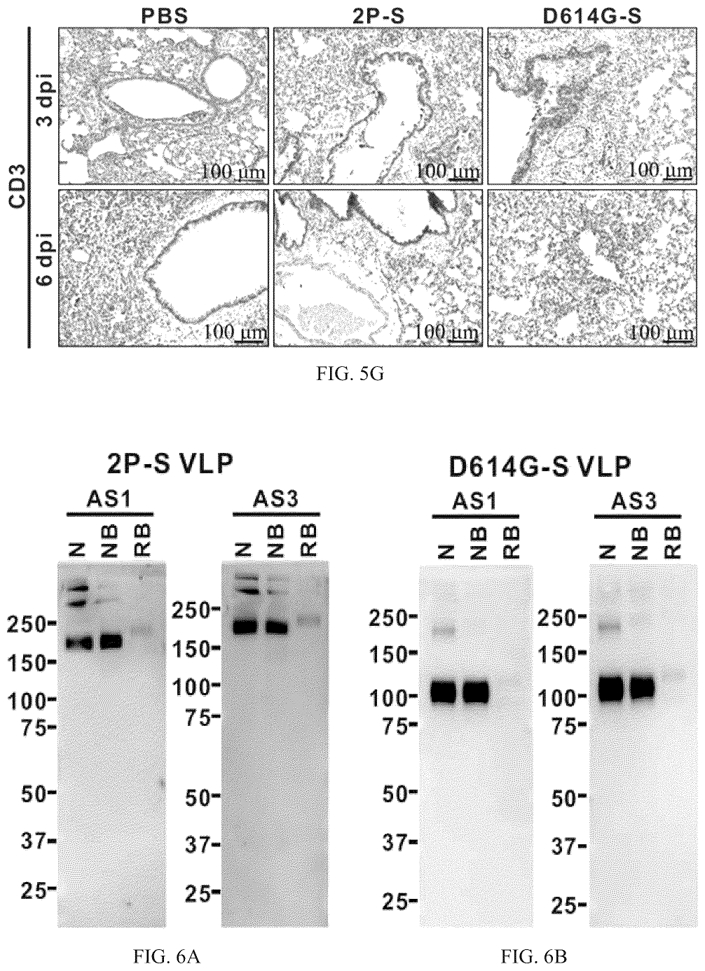

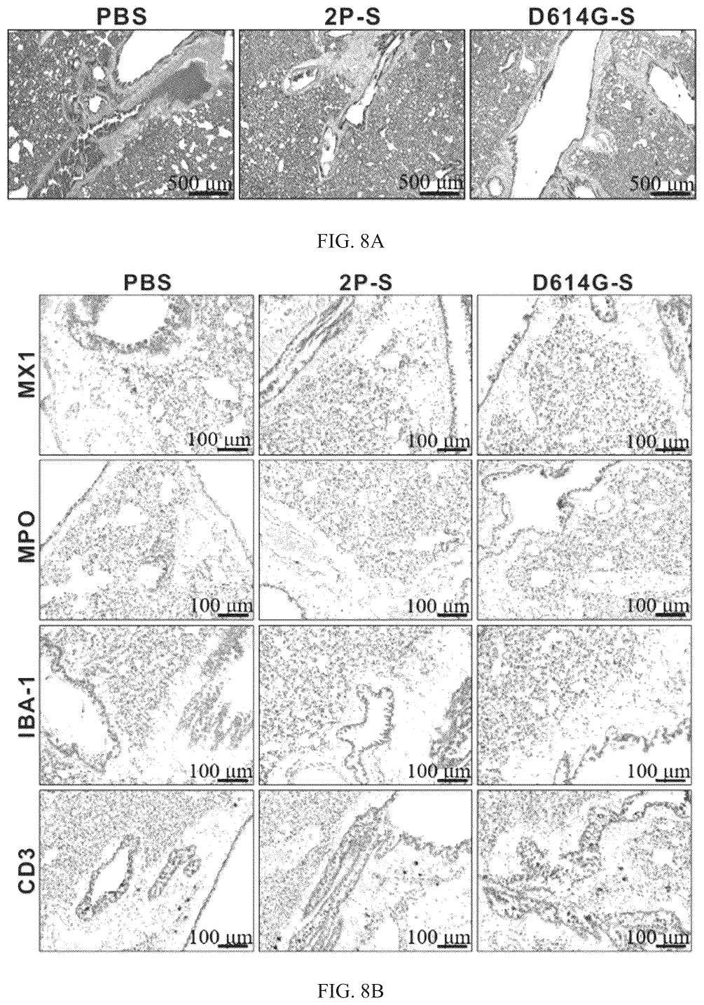

To further investigate the safety and effectiveness of VLP vaccines, we carefully inspected the histopathology and immune response of lung tissue in vaccinated infected and uninfected hamsters at 3 and 6 dpi using H&E staining and immunohistochemistry (IHC). We observed no consolidation, inflammation, or abnormality in the lungs of uninfected hamsters, indicating both VLP vaccines were safe (FIGS. 8A and 8B). Upon SARS-CoV-2 infection, histopathological lung consolidations in the PBS group were initially mild at 3 dpi and progressed to severe at 6 dpi, whereas the consolidations were greatly attenuated in the vaccinated groups (FIGS. 5A and 5B). In the D614G-S lungs, consolidations were mild at 3 dpi and some progressed into moderate coalescing interstitial pneumonia as diffuse lesions at 6 dpi; on the other hand, all 2P-S lungs showed minimal to no evidence of viral interstitial pneumonia throughout 6 dpi except one (n=5) exhibiting moderate consolidations at 6 dpi (FIG. 5B). The in situ viral replication was indicated by IHC of SARS-CoV-N, dark and diffuse SARS-CoV-N in the alveolar edema surrounding the airway was present in PBS lungs at 3 dpi, and subsided but remained visible throughout 6 dpi. In contrast, SARS-CoV-N was limited to solitary cells in 2P-S lungs and small clusters in the D614G-S lungs at 3 dpi and completely clarified at 6 dpi in both vaccinated groups (FIG. 5C). Type I and III interferons are general first-line defenses that responded to viral infection and have the potential to restrain the infection and replication of SARS-CoV-2 (Vanderheiden et al., J Virol 94, 2020). To reflect the interferon response, innate and adaptive immunity in the host lungs, we then inspected the interferon-inducible expression of MX1 and the infiltration of inflammatory cells, MPO⁺ neutrophils, IBA1⁺ macrophages/monocytes, and CD3⁺ T lymphocytes by IHC staining and analysis in the lung specimens of infected hamsters. In the PBS lungs, MX1 was expressed in some alveolar epithelial cells of infected areas at 3 dpi and was widely expressed at lower levels in the thickening area of alveolar septa at 6 dpi, whereas MX1 expression was limited to a low level and density in the infected regions (surrounding the bronchi) of the 2P-S and D614G-S lungs at 3 dpi and diminished in the 2P-S lungs yet remained observable in some hyperplastic pneumocytes in D614G-S lungs at 6 dpi (FIG. 5D). Small clusters of MPO⁺ neutrophils, another professional phagocyte, were also detected in the PBS and D614G-S lungs at 3 dpi, and more clusters were observed in inflammatory regions in the D614G-S lungs at 6 dpi. However, no clusters of macrophages and neutrophils were detected in 2P-S lungs at 6 dpi (FIG. 5E). At 3 dpi, some clusters of macrophages/monocytes were found infiltrated and scattered in the interstitium surrounding airways in both vaccinated groups; however, in the PBS lungs and to a lower level in the D614G-S lungs, the macrophage infiltration accrued and clustered in the pneumonia lesions at 6 dpi, suggesting phagocytosis activity (FIG. 5F). In the PBS lungs, CD3⁺ T lymphocyte infiltrates were dispersed at 3 dpi. Along with the pneumonia development, infiltration of T cells accrued and dispersed in the thickened alveolar interstitium at 6 dpi. In the 2P-S vaccinated subjects, unlike the PBS group, CD3⁺ T cells were primarily locally dispersed in the airway epithelium of the lung and remained unchanged throughout 6 dpi. However, in the D614G-S vaccinated subjects, some CD3⁺ T lymphocytes diffused in the lung interstitium surrounding airways at 3 dpi, further accumulated, and widely spread in the alveoli at 6 dpi, suggesting the activation, expansion, and infiltration of T cells induced by the viral infection (FIG. 5G). These results indicated that the 2P-S and D614G-S vaccines prevented virus infection, spreading, and pneumonia through different mechanisms. The 2P-S vaccine protected hamsters from illness through NAb which blocks the viral binding to host receptors thus efficiently prevents early replication of the virus and subsequent inflammation, whereas the D614G-S vaccine might also be involved in clearing the virus and infected cells using T lymphocyte responses, and antibody-dependent phagocytosis mediated by macrophage/monocyte and neutrophils.

### Example 6 Production of VLPs Derived From the Delta and Omicron Variants of SARS-CoV-2

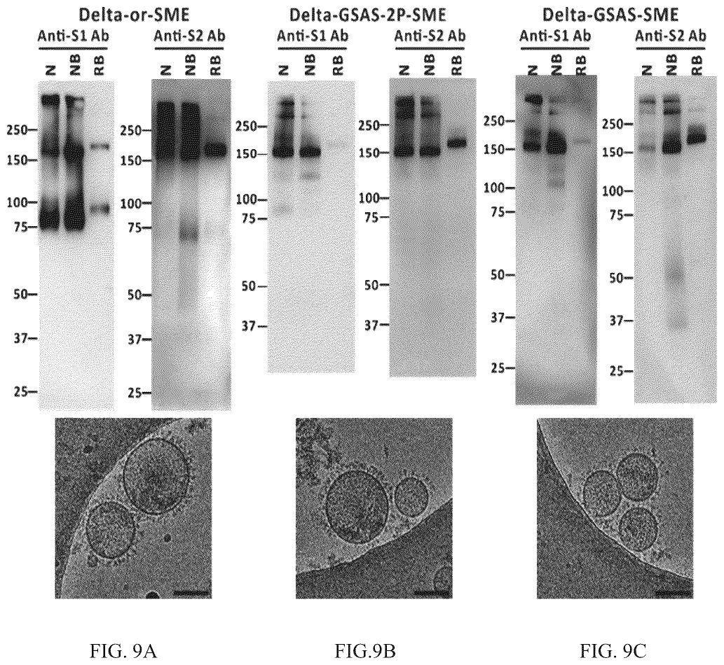

Two series of VLPs based on the spike protein sequences of the Delta and Omicron variants of SARS-CoV-2 were further engineered. Clones of producer cell lines were created and isolated as the method described previously. Co-expression of S protein carrying different mutations as marked, including the original Delta, GSAS-2P, and GSAS mutants, along with M and E proteins produced three different Delta-VLPs. FIGS. 9A to 9C show the characterizations of the S protein by immunoblotting using anti-S1 and anti-S2 antibodies and the spherical morphology of respective VLPs by cryo-EM. As analyzed by dynamic light scattering (DLS), the mean diameters of Delta VLPs were 87.9±0.6 nm, 96.5±0.8 nm, and 114.7±0.4 nm for the original, GSAS-2P, and GSAS mutants, respectively. Similarly, we produced the Omicron VLPs by co-expressing Omicron S carrying different mutations as marked with M and E proteins (FIGS. 9A to 9D). The mean diameters of Omicron VLPs derived from S, M, and E proteins were 114.8±0.8 nm, 109.3±0.4 nm, 110.7±0.7 nm, and 96.4±0.4 nm for the original, 2P-S, RQSR-2P-S, and GSAS-2P-S mutants, respectively.

### Example 7 Over-Expression of S Protein is Sufficient to Drive the Release of VLPs From Animal Cells

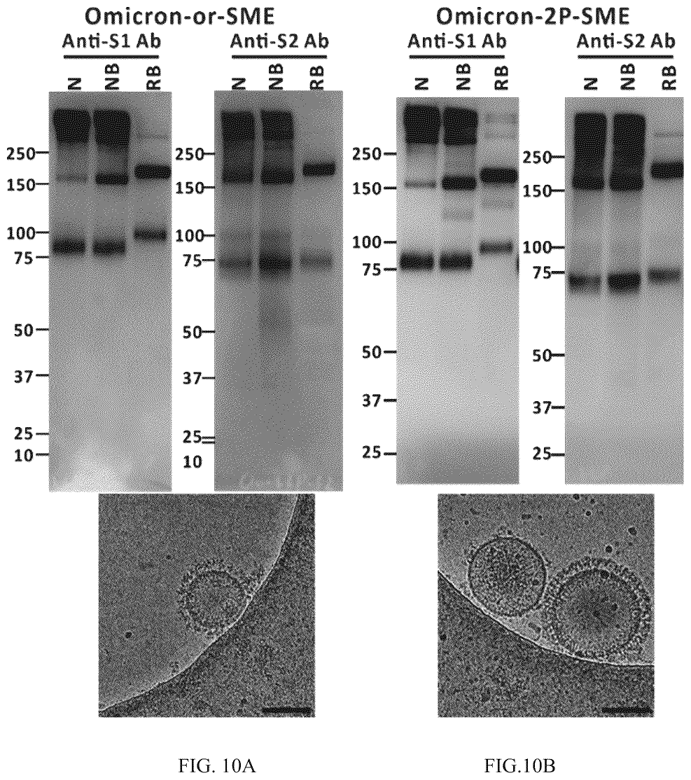

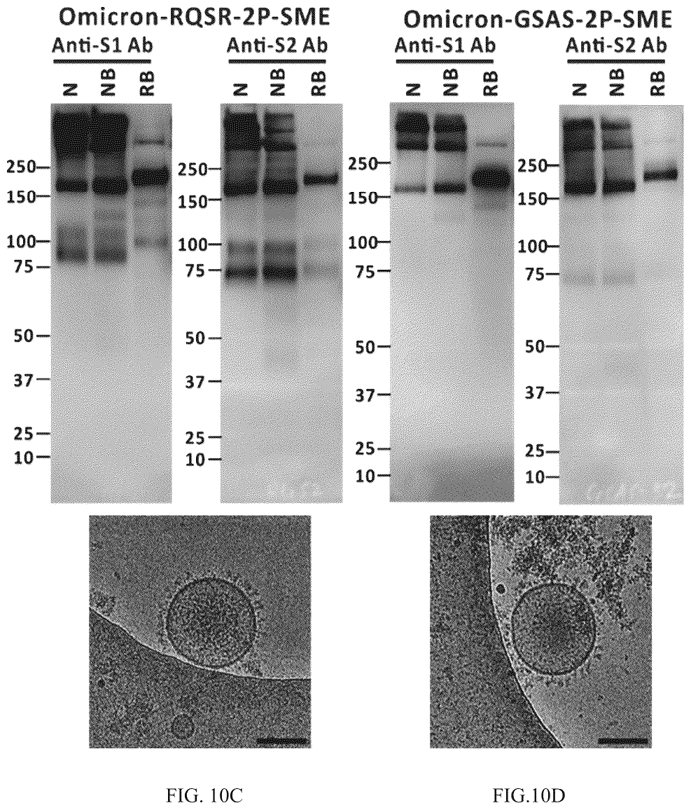

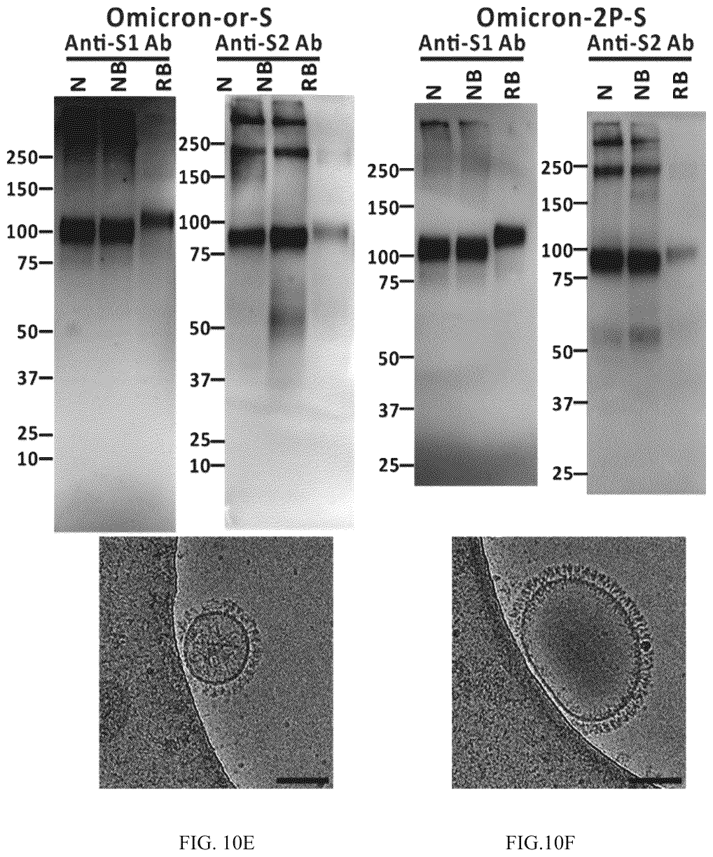

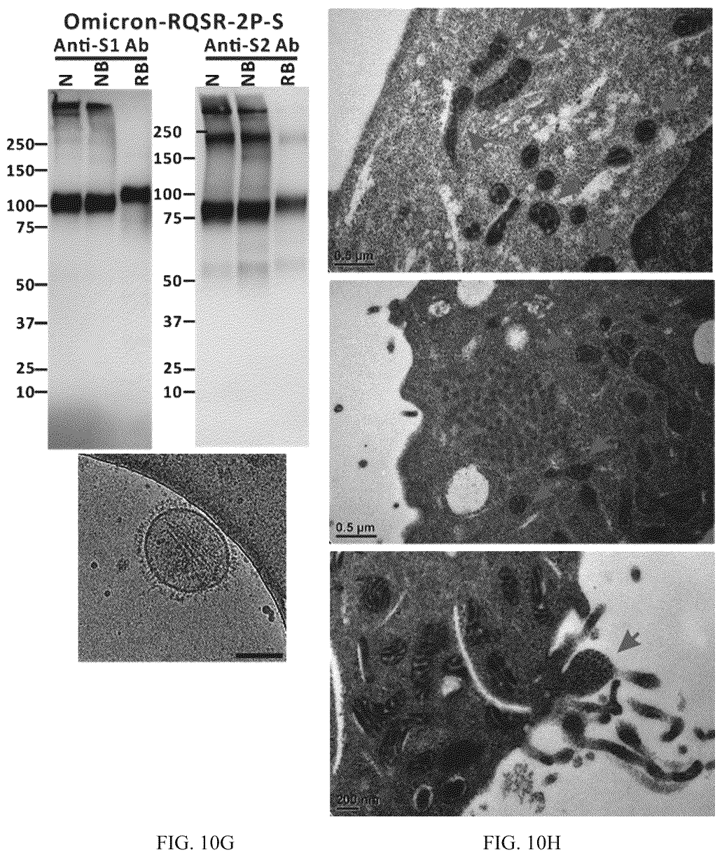

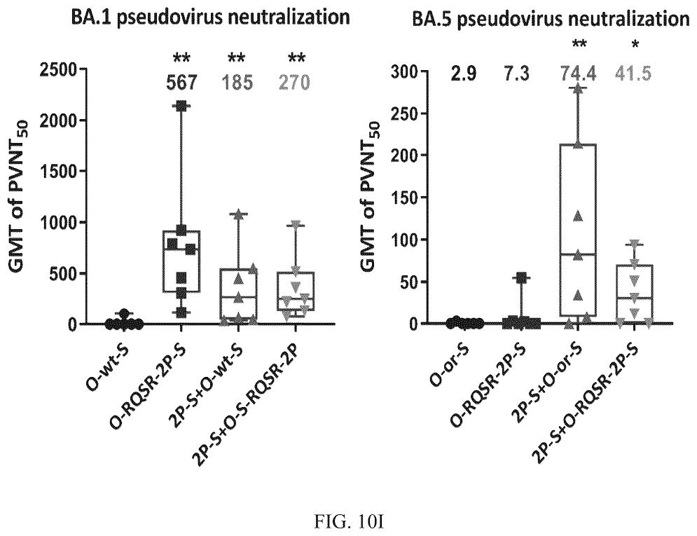

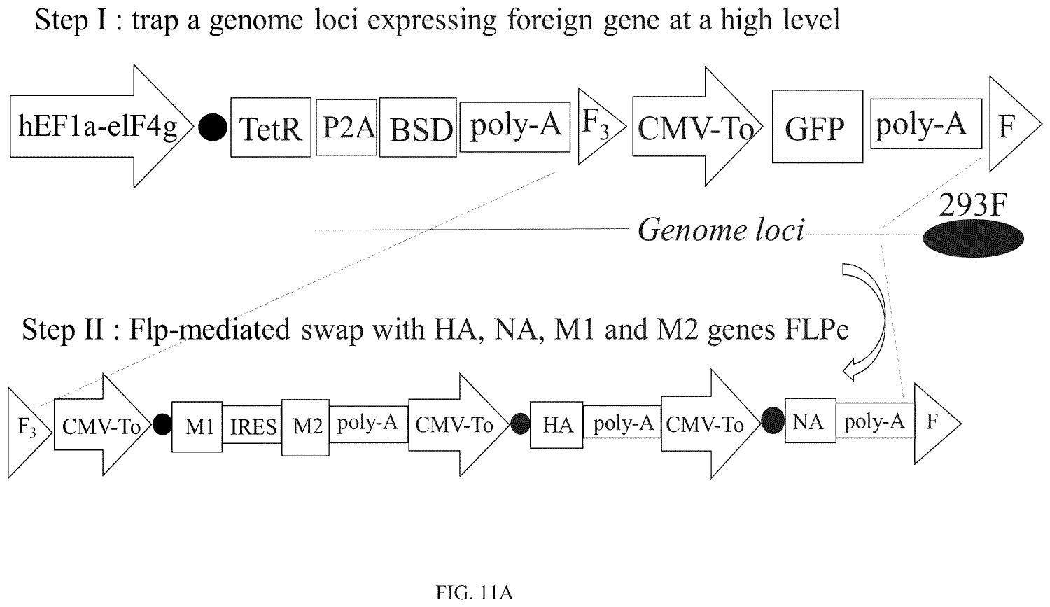

Our proteomic analysis of the VLPs derived from over-expressing the ancestral S, M, and E proteins in 293F cell lines showed very little and almost undetectable levels of M and E proteins. Therefore, we investigated whether expression of the S protein only may drive the budding of VLPs from the transgenic cells. Interestingly, over-expression of S protein only in the 293F cell line also produced VLPs and released in the culture medium, including the ancestral S (wild-type) (SEQ ID NO: 10) and its GSGS-2P-S mutant (SEQ ID NO: 12), the Omicron S (original) (SEQ ID NO: 14), and Omicron 2P-S and RQSR-S (SEQ ID NO: 16) mutants (FIGS. 10E to 10G). After purification, the mean diameters of Omicron VLPs derived from Omicron S protein only were 105.5±0.4 nm, 114.1±1.0 nm, and 124.4±0.5 nm for the original, 2P-S, and RQSR-2P-S mutants, respectively (FIGS. 10E to 10G). This is the first evidence that over-expression of only the S protein of a coronavirus is sufficient to drive the budding of coronavirus-like particles within the cell and productively released the VLPs in the extracellular space and cell culture medium (FIG. 10H).

### Example 8 VLPs of the Omicron Variant Have a Reduced Ability to Elicit Neutralizing Antibodies

The assessment of candidate vaccines has been performed by immunization using a prime-boost (2-shot) vaccine regimen in mice models. The VLPs derived from the S protein of Delta and Omicron variants and mutants formulated with an AS03-like adjuvant (a Squalene-Oil-in-water preparation provided by RuenHuei Biopharmaceuticals Inc.) exhibited much lower immunogenicity compared with the 2P-S VLP derived from the ancestral S protein sequence. Therefore, raising Omicron VLPs dose to five folds (containing 3.75 μg S protein/mouse) and with/without formulation of the ancestral 2P-S VLP (containing 0.75 μg S protein/mouse) into a divalent vaccine are our strategies in the animal experiment for a new vaccine or booster vaccine. Female K18-hACE2 [B6.Cg-Tg (K18-ACE2) 2Prlmn/J] mice (n=7 or 8, 8 weeks old) were subcutaneously injected with monovalent Omicron S VLP (wild-type BA. 1 or its RQSR-2P mutant), and their combinations with the ancestral 2P-S VLP (1:5 W/W) admixed with AS03-like adjuvant (1:1, V/V) as respective divalent vaccines on day 0 and 21 and bled on day 35. Specific IgG titers in the pooled antisera against the Omicron-wt-S VLP reached GMT: 2×10⁵ in ELISA. The neutralization potency of immune sera against Omicron BA.1 pseudotyped lentiviruses indicated by 50% pseudovirus neutralization titers (PVNT₅₀) of O-RQSR-2P-S and the two divalent vaccines were significantly higher than the O-wt-S. The PVNT₅₀ against BA.5 of vaccinated mice in the divalent vaccines (Omicron 2P-S and either Omicron O-wt-S or O-RQSR-2P-S) elicit significantly higher titers of neutralization than the monovalent groups, suggesting a contribution of cross-protective antibodies due to the ancestral 2P-S VLP (FIG. 10I). In contrast, these VLPs formulated with MF59-like adjuvant produced lower titers of neutralization (data not shown). Our data suggest the damping immunogenicity of Omicron VLP (containing 3.75 μg S protein/mouse) requires formulation with more potent adjuvants, such as AS03-like Squalene-Oil-in-water emulsion or others, to stimulate high-titer neutralization antibodies. The data have demonstrated that VLP displaying Omicron S with RQSR-2P mutations can be effective to comprise strain-matched vaccine.

### Example 9 Production of H5N2 Influenza Virus-Like Particle

### Trapping of a Chromosomal Locus That Allows the Highest Level of Inducible Foreign Gene Expression in Human FreeStyle 293F and Insect High-Five Cell Lines

To produce VLPs of the H5N2 influenza virus (A/duck/Taiwan/01006/2015/H5N2) using human 293F cell line and insect High-Five cell line, we constructed plasmids designed for doxycycline-inducible expression of GFP in mammalian and insect expression systems. Particularly, the promoters to drive insect cell expression need to be modified from the gene promoter of Orgyia pseudotsugata multicapsid nucleopolyhedrosis virus (OpMNPV) immediate-early 2 (IE2) into a doxycycline-inducible strong promoter and from the gene promoter of Antheraea pernyl actin-A1 into a doxycycline-inducible weak promoter. In principle, constitutively expressed Tet-repressor holds back the expression from IE2 and A. pernyl actin-A1 promoters through binding to the tet operator 2 (TetO2) inserted downstream of the TATA-box until the Tet-repressor is unleashed by doxycycline treatment. We then made the founder cells by stable transfection in FreeStyle 293F using FreeStyle MAX reagent. Cell presenting phenotypes with low basal and high induction of GFP expression were enriched twice by flowcytometry-assisted cell sorting (FACS). Single-cell clones were isolated from the enriched cells to obtain the best founder cell lines.

### Engineering of the Donor Plasmid Harboring an FRT-Flanked Cluster of Several Transgene Cassettes, CMVITO Promoter-Driven, and Expressing 3 or 4 Viral Structural Proteins

It is known that coexpression of S, M, E, and/or N proteins in the same mammalian cells will release VLPs into the cell culture medium. Therefore, we constructed the CMV/TO driven M-IRES-E, S, with/without N, distributed into 2 or 3 tandem genes in the same plasmid. The entire cluster of transgenes is flanked by two FRT sites (F and Fn) to be the target cassette of FLPe flippase. The sequence of H5 protein is shown as SEQ ID NO: 18; the sequence of N2 protein is shown as SEQ ID NO: 20; the sequence of M1 protein is shown as SEQ ID NO: 22); and the sequence of M2 protein is shown as SEQ ID NO: 24.

### “Gene swapping” the Donor Cassettes Through Cotransfection With FLPeRrecombinase to Accomplish Site-Specific Insertion of all Transgene

The donor plasmid and FLPe expression plasmid were cotransfected into founder cell lines derived from human FreeStyle 293F and insect High-Five cell lines, respectively. Transfected cells were expanded and enriched by loss-of-GFP expression using FACS. The enriched cells were sorted again by loss-of-GFP and gain-of-HA expression in the H5N2 influenza VLP. Single clones of gene swapped cells were isolated and screened clones with the highest-level expression of HA and the background level of GFP expression upon doxycycline induction for 2 days. HA should exhibit low-level staining in the absence of doxycycline. Using this “swift multi-gene overexpression system”, we generated stable VLP-producing 293F clones.

### Production and Purification of H5N2 Influenza VLP

A stable VLP-producing clone (#71) was expanded to 2×10⁶ cells/mL density in serum-free suspension cell culture in FreeStyle™ 293 Expression Medium. Expression and release of VLPs were initiated by adding doxycycline to the cell culture at 1 μg/mL working concentration. Doubling time of 293F cells at a healthy exponential growth phase ranged from 22 to 28 hours. Cell densities could be maintained at up to 3×10⁶ cells/mL in a shaker or spinner culture and up to 4×10⁶ cells/mL in bioreactor culture. The culture medium was refreshed every 3 days, and VLPs were collected from the conditioned medium, filtered through 0.45 μm to remove cell debris and other large aggregates, concentrated by Tangential Flow Filtration (TFF), and finally purified by a two-step sucrose-gradient (30% and then 40%-60%) ultracentrifugation or 1-step column chromatography Capto Core 700 multimodal (MMC) and diafiltration, or 2-step column chromatography using Capto DeVirS and Capto Core 700 to eliminate most biological impurities.

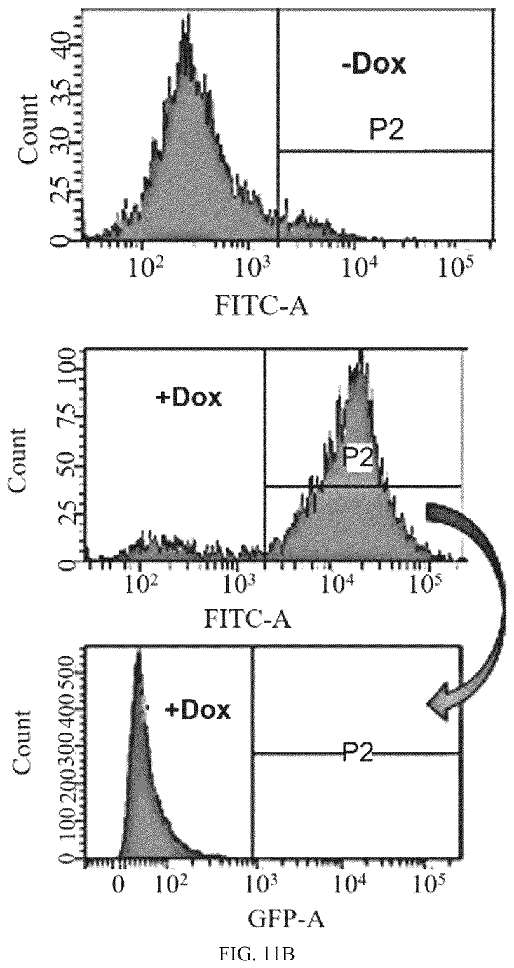

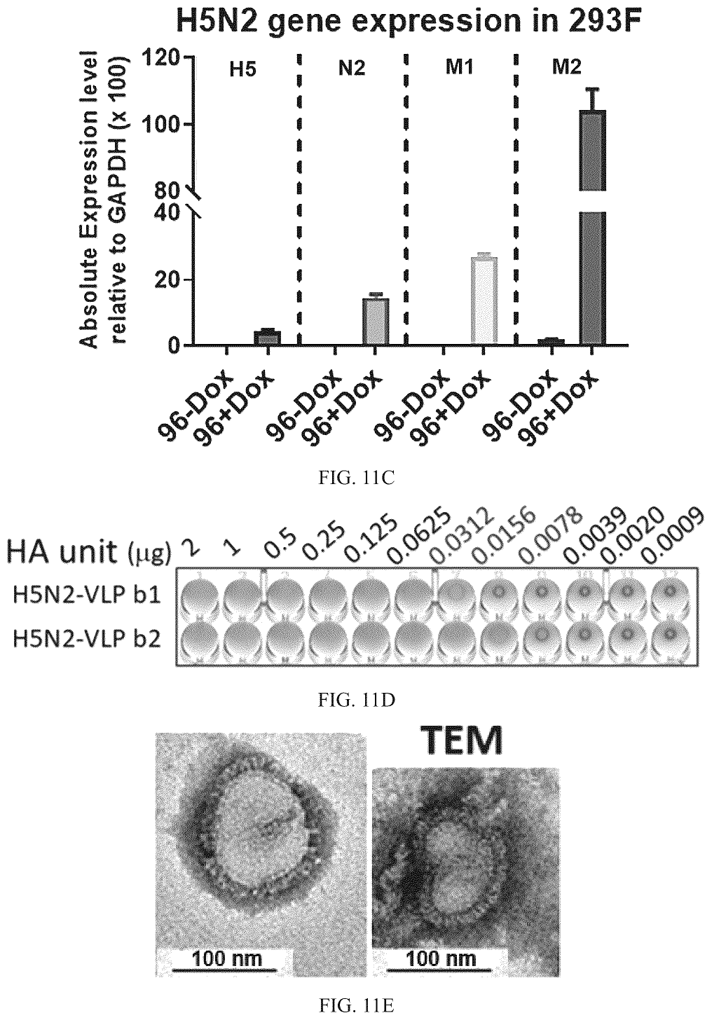

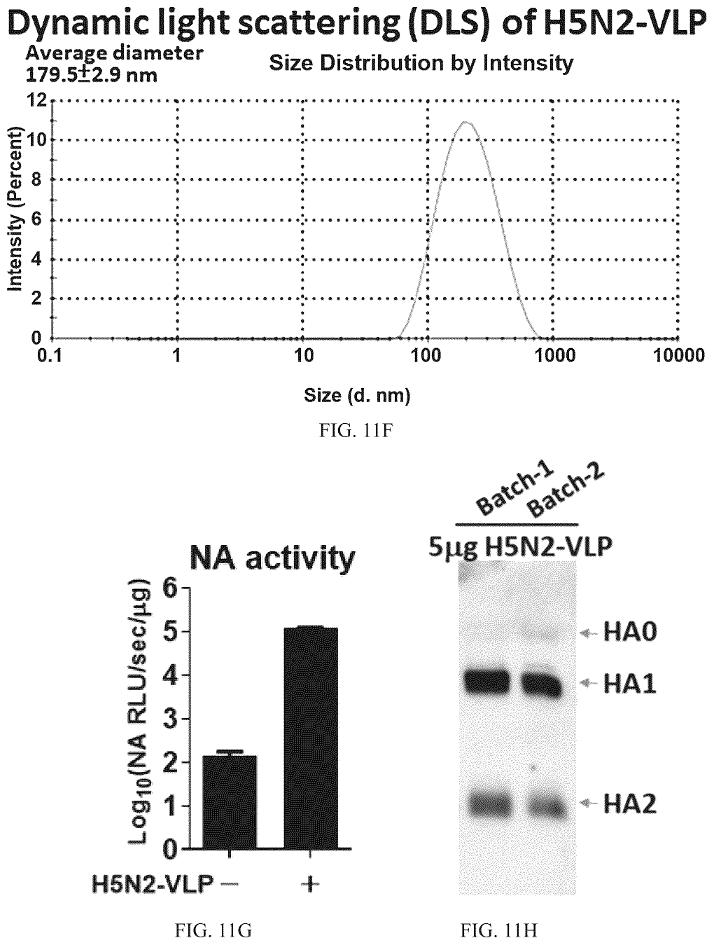

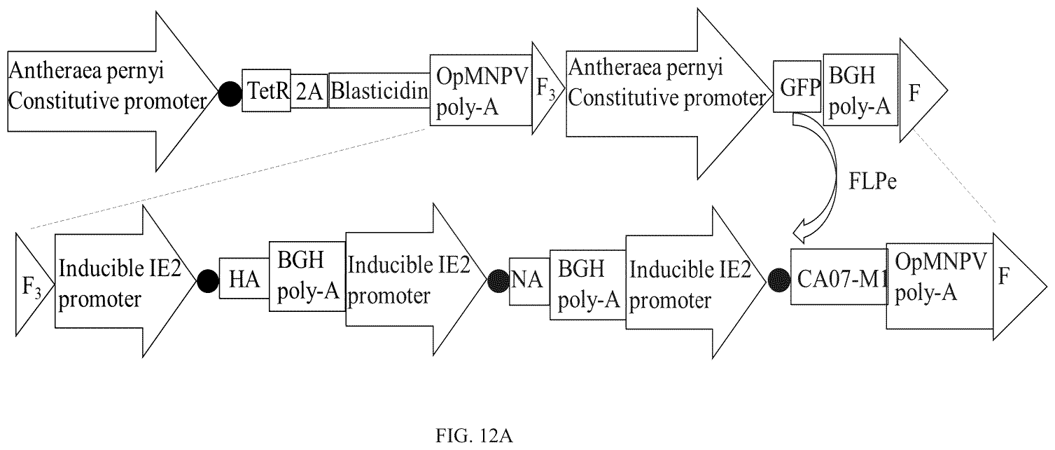

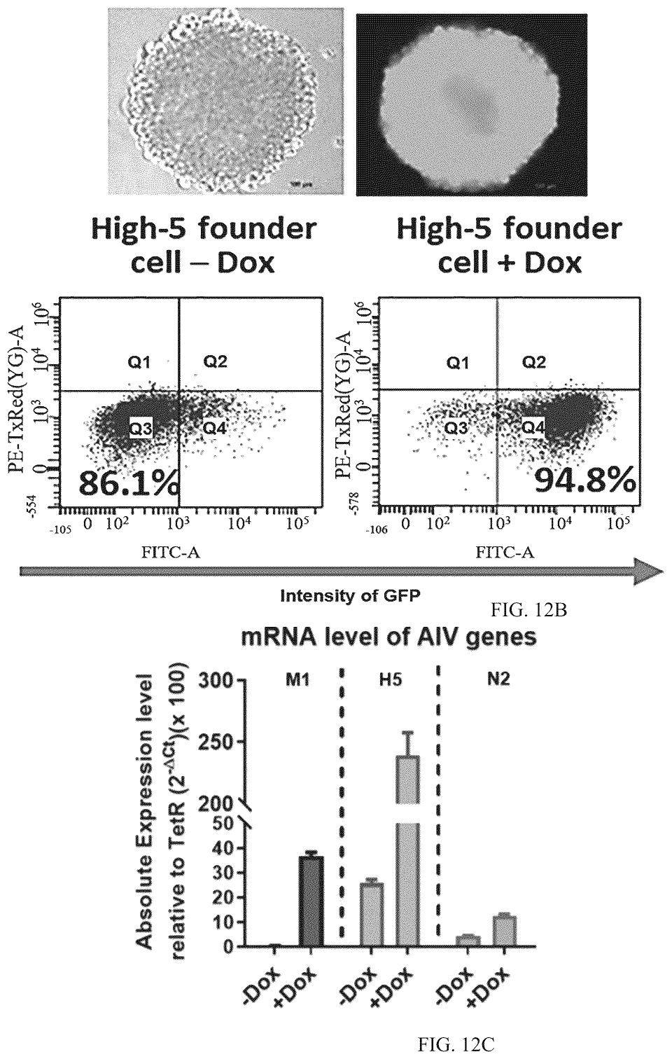

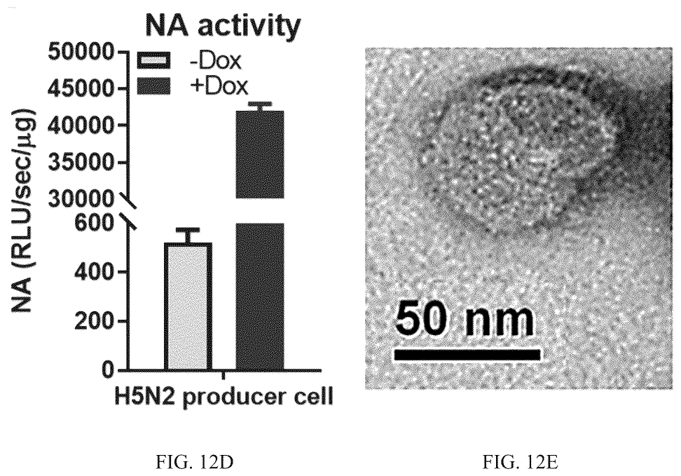

The results are shown in FIGS. 11A to 11H. The H5N2 influenza VLPs were successfully produced and purified.

While the present invention has been described in conjunction with specific embodiments as set forth, many alternatives thereto and modifications and variations thereof will be apparent to those of ordinary skill in the art. All such alternatives, modifications and variations are regarded as falling within the scope of the present invention.

1. An animal cell stably expressing a virus-like particle (VLP), comprising an inducible expression cassette of one or more site-specific recombinant VLP genes.

2. The animal cell of claim 1, wherein the animal cell is an insect cell or a mammalian cell.

3. (canceled)

4. The animal cell of claim 1, wherein the virus-like particle comprises a coronaviral structural protein or an influenza viral structural protein.

5. The animal cell of claim 4, wherein the coronaviral structural protein is a structural protein of SARS-CoV-2 (COVID-19).

6. (canceled)

7. (canceled)

8. The animal cell of claim 5, wherein the coronaviral structural protein comprises a spike protein (S) and the spike protein is a native D614G spike protein (SEQ ID NO: 6), a di-proline mutant spike protein (2P-S) (SEQ ID NO: 8), a D614G-S mutant spike protein, Delta-or spike protein (SEQ ID NO: 10), Delta-GSAS 2P spike protein (SEQ ID NO: 12), Omicron-or spike protein (SEQ ID NO: 14), or Omicron-GSAS 2P spike protein (SEQ ID NO: 16).

9. The animal cell of claim 8, wherein the spike protein is coded by a DNA sequence selected from the group consisting of SEQ ID NOs: 7, 9, 11, 13, 15, and 17.

10. The animal cell of claim 4, wherein the influenza viral structural protein is a structural protein of H5N2 or H3N2 influenza virus.

11. (canceled)

12. The animal cell of claim 4, wherein the influenza viral structural protein is selected from the group consisting of H5 protein (SEQ ID NO: 18), N2 protein (SEQ ID NO: 20), M1 protein (SEQ ID NO: 22), and M2 protein (SEQ ID NO: 24).

13. The animal cell of claim 12, wherein the influenza viral structural protein is coded by a DNA sequence selected from the group consisting of SEQ ID NOs: 19, 21, 23 and 25.

14. The animal cell of claim 1, which is established by stably transfecting a target cassette which comprises an inducible tetracycline-inducible promoter-reporter or a doxycycline-inducible promoter-reporter of Flp/FRT recombination system and a stably-expressed tetracycline repressor cassette; and gene swapping the target cassette and tetracycline repressor cassette through cotransfection with FLPe recombinase to accomplish site-specific insertion of all VLP genes.

15. The animal cell of claim 1, wherein the inducible expression cassette comprises a tetracycline-inducible promoter or a doxycycline-inducible promoter.

16. (canceled)

17. The animal cell of claim 1, which stably expresses a tetracycline repressor cassette, wherein the tetracycline repressor cassette comprises a tetracycline repressor gene, and a blasticidin S-resistance gene connected by a self-cleaving 2A peptide derived from porcine teschovirus-1.

18. The animal cell of claim 1, wherein the inducible expression cassette comprises CMV/TO, Orgyia pseudotsugata multicapsid nucleopolyhedrosis virus (OpMNPV) immediate-early 2 (IE2) or Antheraea pernyl actin-A1 promoter.

19. The animal cell of claim 17, wherein the tetracycline repressor cassette is EF1a/eIF4g-pCI-TetR-P2A-BSD cassette.

20. A method for manufacturing a virus-like particle, comprising culturing the animal cell of claim 1 and harvesting the virus-like particle.

21. A virus-like particle manufactured by the method of claim 20.

22. A vaccine composition comprising an immunologically effective amount of the virus-like particle of claim 21 and optionally an adjuvant.

23. (canceled)

24. A method for preventing viral infection, producing antibodies specific to the VLPs, preventing viral replication, or alleviating symptoms due to the viral infection in a subject comprising administering the vaccine composition of claim 22 to the subject.

25. (canceled)

26. The method of claim 24, wherein the viral infection is SARS-CoV-2 infection or H5N2 or H3N2 influenza viral infection.

27. (canceled)

28. (canceled)

29. The method of claim 24, wherein the antibodies specific to the VLPs is further harvested from the subject.
